(19) United States
(12) Patent Application Publication
(10) Pub. No.: US 2012/0121554 A1
(43) Pub. Date: May 17, 2012

(54) HMG-COA SECONDARY METABOLITES AND USES THEREOF

(75) Inventor: Kirk Pappan, Abilene, KS (US)

(73) Assignee: EDENSPACE SYSTEMS CORPORATION, Manhattan, KS (US)

(21) Appl. No.: 13/318,452

(22) PCT Filed: Apr. 30, 2010
(86) PCT No.: PCT/US10/33286

§ 371 Date: Jan. 19, 2012

Related U.S. Application Data
(60) Provisional application No. 61/174,966, filed on May 1, 2009.

(51) Int. Cl. A61K 35/12; A61P 9/12; A61P 7/00; A61P 9/10; A61P 9/00; A61P 37/06; A61P 37/00; A61P 25/28; A61P 19/02; A61P 35/00; A61P 3/00; A61P 25/00; A61P 3/10; A61P 1/00; A61P 5/16; A61P 19/04; A61P 17/06; A61P 1/16; A61P 17/00; A61P 1/04; A61P 29/00; C12Q 1/02; C12Q 1/26; G01N 30/00; H01J 49/26; A61K 31/194

(52) U.S. Cl. 424/93.7; 514/574; 435/29; 435/25; 73/61.43; 73/61.52; 250/282

(57) ABSTRACT

The present invention is directed, among other things, to using secondary metabolites in the mevalonate pathway (such as, for example, HMG) and/or structurally related compounds to mediate biological activities (e.g., for therapeutic applications) and/or as diagnostic agents. In some embodiments, the biological activities comprise one or more pleiotropic effects of statins (such as, for example, angiogenesis, promoting vascular function, anti-inflammatory action, immunomodulation, etc.). Also provided are methods of screening for mevalonate pathway secondary metabolites, methods of producing HMG, and methods of diagnosing comprising measuring amount of mevalonate pathway secondary metabolites.

### RELATED APPLICATION

The present application claims priority to, and benefit of, U.S. provisional patent application no. U.S. Ser. No. 61/174,966 (filed May 1, 2009), the entire contents of which are herein incorporated by reference.

### BACKGROUND

Statins are a class of drugs used by millions of patients worldwide to lower their LDL (low density lipoprotein) cholesterol levels. Statins are also known as HMG-CoA reductase inhibitors because they act by inhibiting the enzyme HMG-CoA reductase, the rate-limiting enzyme of the mevalonate pathway of cholesterol synthesis. In the mevalonate pathway (see FIG. 1), HMG-CoA (3-hydroxy-3-methylglutaryl-CoA) is converted to mevalonic acid by HMG-CoA reductase. Mevalonic acid can then undergo a series of reactions leading to the production of dimethylallyl pyrophosphate (DMAPP) and isopentyl pyrophosphate (IPP), which serve as a basis for the biosynthesis of molecules (including steroids) used in a variety of processes.

In addition to lowering cholesterol levels, statins have pleiotropic effects that are unrelated to cholesterol. Such pleiotropic effects impact a variety of biological and disease processes and include improved vascular function, angiogenesis, enhanced recovery from ischemia, reduced inflammation, and modified immune cell responses.

### SUMMARY

The present invention encompasses the recognition that a mechanistic understanding of statin pleiotropy would greatly enhance the therapeutic potential of statins and related molecules. Described herein is an alternate biochemical process by which HMG-CoA is metabolized into HMG (3-hydroxy-3-methylglutaric acid) when statins block its conversion to mevalonic acid. The present invention further encompasses the discovery that HMG reduces inflammatory autoimmune and inflammatory responses. Without wishing to be bound by any particular theory, the present invention proposes that HMG may mediate certain pleiotropic effects of statins and specifically that HMG production secondary to statin inhibition may account for or at least participate in the pleiotropic effects observed with statins. The present invention therefore demonstrates that secondary metabolites in the mevalonate pathway may have therapeutic value.

Disclosed herein are novel systems for treating and diagnosing a variety of diseases or conditions using secondary metabolites (such as HMG) or derivatives thereof in the mevalonate pathway. Disclosed herein also are systems for identifying and/or characterizing therapeutic and/or diagnostic activities of secondary metabolites in the mevalonate pathway.

In various embodiments, provided are methods of producing HMG, methods of screening for secondary metabolites in the mevalonate pathway with beneficial effects, methods of promoting blood vessel formation and/or vascular function using an HMG agent, methods of reducing inflammation and/or modulating the immune system using an HMG agent, and methods of diagnosing using an HMG agent.

### BRIEF DESCRIPTION OF THE DRAWING

FIG. 1 depicts major components of the mevalonate pathway.

FIG. 2 depicts chemical structures of HMG (3-hydroxy-3-methylglutarate), HMG-CoA, mevalonic acid, citrate, and hydroxycitrate.

FIG. 3 depicts synthetic routes and routes of disposal of HMG-CoA.

FIG. 4 depicts a reaction scheme to generate a variety of 3-alkyl-3-hydroxyglutaric acid derivatives of HMG having inhibitory activity against HMGR. Shown are base structures of 3-alkyl-3-hydroxyglutaric acid derivatives listed in Table 1. ^{a} Allylmagnesium bromide. ^{b}O₃; H₂O₂, H+. ^{c}K₂CO₃-MeI or Et I, DMF. ^{d}DCCD or Ac₂O, A. ^{e}(CH₃)₂NCH₂CH₂OH or EtOH, Py. ^{f}NH₃; MeI-K₂CO₃. ^{g}Py, water. ^{h}LAH. ⁱCrO₃.

FIG. 5 depicts a schematic showing a proposed mechanism for pleiotropic effects of statins. In the depicted schematic, HMG is produced secondarily to HMG-CoA reductase inhibition.

FIG. 6 depicts a schematic showing a proposed mechanism for effects of HMG in vivo.

FIG. 7 depicts serum levels of cytokines and chemokines in various treatment groups (PBS only control, PLP-BPI-treated, and HMG-treated) 14 days after immunization with PLP₁₃₉₋₁₅₁/CFA to induce EAE. Data represent the mean±standard deviation. Statistical significance was determined by one way Analysis of Variance followed by post-hoc testing using the Tukey test. Bars labeled with the same letter did not differ significantly (p>0.05) from one another, whereas bars labeled with different letters showed statistically significant differences (p<0.05).

### DEFINITIONS

Throughout the specification, several terms are employed that are defined in the following paragraphs.

As used herein, the terms “about” and “approximately,” in reference to a number, is used herein to include numbers that fall within a range of 20%, 10%, 5%, or 1% in either direction (greater than or less than) the number unless otherwise stated or otherwise evident from the context (except where such number would exceed 100% of a possible value).

As used herein, the term “administer” means giving something (such as, for example a therapy, treatment, agent, compound, and/or dose thereof) to an individual. Routes of administration include, but are not limited to, topical (including epicutaneous, enema, eye drops, ear drops, intranasal, vaginal, etc.), enteral (including oral, feeding tube, rectal, etc.), parenteral (including intravenous, intraarterial, intramuscular, intracardiac, subcutaneous, intraosseous, intradermal, intrathecal, intraperitoneal, transdermal, transmucosal, inhalational, infusion, etc.), epidural, and intravitreal.

As used herein, the phrase “anti immuno-inflammatory agent” refers to an agent that reduces or inhibits, partially or totally, immediately or after a delay, inflammation or one of its manifestations as well as other immune responses.

As used herein, the phrase “anti-inflammatory agent” refers to an agent that reduces or inhibits, partially or totally, immediately or after a delay, inflammation or one of its manifestations, for example migration of leucocytes by chemotaxis. An MHC Class II-mediated anti-inflammatory agent is an anti-inflammatory agent whose key action on the immune system involves molecules of MHC class II.

As used herein, the phrase “biological activity” refers effect(s) of something (e.g., a drug, a compound, a substance, a protein, etc). on living matter. In some embodiments, the effect is beneficial. In some embodiments, the effect is adverse. In some embodiments, the activity is dosage-dependent. In some such embodiments, effects may range from beneficial to adverse depending on dosage.

The term “biologically active”, when used herein to characterize a derivative or analog of a compound, refers to a molecule that shares sufficient structural similarity with a starting compound to exhibit similar or identical properties to the starting compound (e.g., ability to promote blood vessel formation, promote vascular function, reduce inflammation, modulate the immune system, etc.).

The term “combination therapy”, as used herein, refers to those situations in which two or more different pharmaceutical agents are administered in overlapping regimens so that the subject is simultaneously exposed to both agents.

A “dosing regimen”, as that term is used herein, refers to a set of unit doses (typically more than one) that are administered individually separated by periods of time. The recommended set of doses (i.e., amounts, timing, route of administration, etc.) for a particular pharmaceutical agent constitutes its dosing regimen.

As used herein, the terms “effective amount” and “effective dose” refer to any amount or dose of a compound or composition that is sufficient to fulfill its intended purpose(s), i.e., a desired biological or medicinal response in a tissue or subject at an acceptable benefit/risk ratio. For example, in certain embodiments of the present invention, the purpose(s) may be: to promote angiogenesis, promote vascular function, reduce inflammation, modulate the immune system, etc. The relevant intended purpose may be objective (i.e., measurable by some test or marker) or subjective (i.e., subject gives an indication of or feels an effect). A therapeutically effective amount is commonly administered in a dosing regimen that may comprise multiple unit doses. For any particular pharmaceutical agent, a therapeutically effective amount (and/or an appropriate unit dose within an effective dosing regimen) may vary, for example, depending on route of administration, on combination with other pharmaceutical agents, etc. In some embodiments, the specific therapeutically effective amount (and/or unit dose) for any particular patient may depend upon a variety of factors including the disorder being treated and the severity of the disorder; the activity of the specific pharmaceutical agent employed; the specific composition employed; the age, body weight, general health, sex and diet of the patient; the time of administration, route of administration, and/or rate of excretion or metabolism of the specific pharmaceutical agent employed; the duration of the treatment; and like factors as is well known in the medical arts.

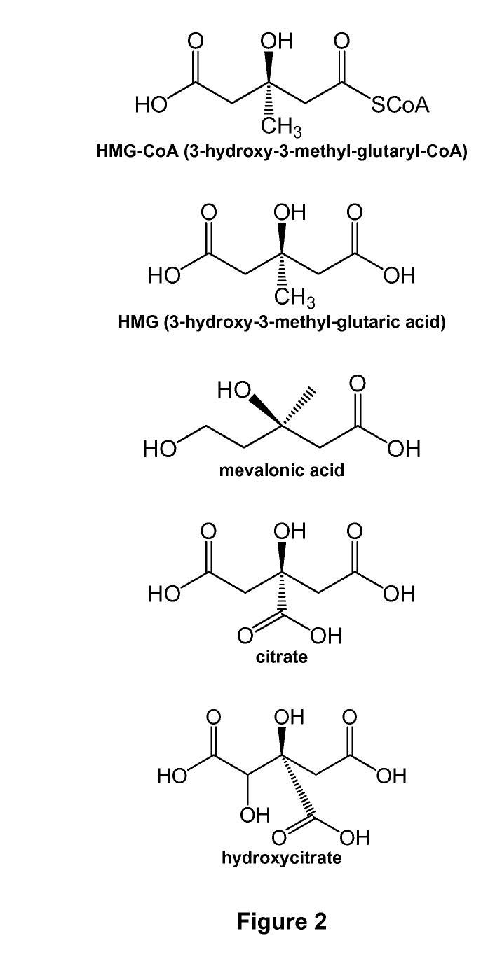

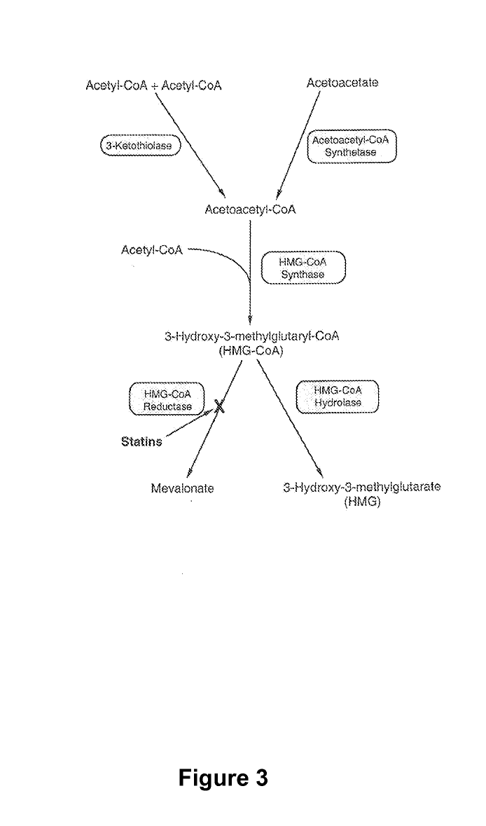

As used herein, the term “HMG” is used interchangeably with “3-hydroxy-3-methylglutarate” and refers to a compound having the structure shown in FIG. 2. HMG is a metabolite produced by the hydrolysis of HMG-CoA by HMG CoA hydrolase (see FIG. 3) and is maintained in the micromolar range in the plasma of healthy adults (Lippe et al. 1987). HMG is listed in the CAS (Chemical Abstracts Service) registry as compound number 503-49-1. HMG is also known as Meglutol, Mevalon, beta-hydroxy-beta-methylglutarate, and 3-hydroxy-3-methylpentanedioic acid.

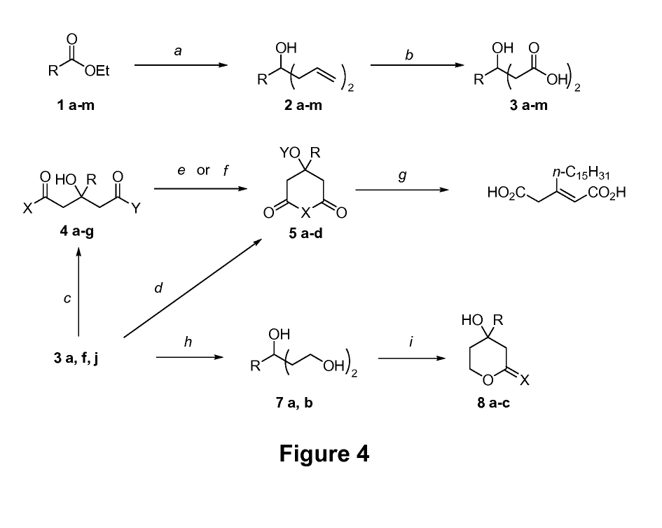

As used herein, the term “HMG agent” is used interchangeably with “3-hydroxy-3-methylglutaric acid agent” and refers to 3-hydroxy-3-methylglutaric acid (HMG) and structurally related compounds (e.g., analogs, derivatives, etc.). In some embodiments, such structurally related compounds include alkyl derivatives such as 3-alkyl-3-hydroxyglutaric acids. (See, for example, Baran et al. 1985. “3-Alkyl-3-hydroxyglutaric Acids: A New Class of Hypocholesterolemic HMG CoA Reductase Inhibitors.” J. Med. Chem. 28:597-601, the contents of which are hereby incorporated by reference in their entirety.) Examples of 3-alkyl-3-hydroxyglutaric acids include, without limitation, 3-n-pentadecyl-3-hydroxyglutaric acid, and other compounds having core structures depicted in FIG. 4 and whose substituent groups are listed in Table 1. In some embodiments, HMG agents inhibit HMG-CoA reductase. In some embodiments, HMG agents mediate one or more pleiotropic effects of statins.

TABLE 1
HMGR inhibition by 3-alkyl-3-hydroxyglutaric acids
and their derivatives (from Baran et al.)

| | | | | in vitro inhibition | |
|---|---|---|---|---|---|
| | | | | of HMGR | |
| Com- | | | | | IC₅₀^{a} |
| pound | R | X | Y | @10⁻³ M | (μM) |
| 3a | CH₃ | | | | |
| 3b | CH₃CH₂ | | | 29 | |
| 3c | CH₃(CH₂)₂ | | | 37 | |
| 3d | (CH₃)₂CH | | | 10 | |
| 3e | CH₃(CH₂)₅ | | | 5 | |
| 3f | CH₃(CH₂)₉ | | | 86 | 100 |
| 3g | CH₃(CH₂)₁₂ | | | 97 | 50 |
| 3h | CH₃(CH₂)₁₃ | | | 95 | 130 |
| 3j | CH₃(CH₂)₁₄ | | | 99 | 50 |
| 3k | CH₃(CH₂)₁₅ | | | 90 | 100 |
| 3l | CH₃(CH₂)₁₆ | | | 85 | 100 |
| 3m | CH₃(CH₂)₁₈ | | | 86 | 350 |
| 4a | CH₃ | OC₂H₅ | OC₂H₅ | 0 | |
| 4b | CH₃ | OCH₃ | NH₂ | 0 | |
| 4c | CH₃(CH2)₉ | OC₂H₅ | OC₂H₅ | 0 | |
| 4d | CH₃(CH₂)₁₄ | OCH₃ | OCH₃ | −1 | |
| 4e | CH₃(CH₂)₁₄ | OC₂H₅ | OC₂H₅ | 36 | >1000 |
| 4f | CH₃(CH2)₁₄ | OH | OC₂H₅ | 81 | 900 |
| 4g | CH₃(CH2)₁₄ | OH | O(CH₂)₂N(CH₃)₂ | 0 | |
| 5a | CH₃ | O | | 0 | |
| 5b | CH₃ | NH | | 50 | |
| 5c | CH₃(CH₂)₁₄ | O | | 80 | 250 |
| 5d | CH₃(CH₂)₁₄ | O | COCH₃ | −9 | |
| 6 | CH₃(CH₂)₁₄ | | | 0 | |
| 7a | CH₃(CH₂)₉ | | | 0 | |
| 7b | CH₃(CH₂)₁₄ | | | 0 | |
| 8a | CH₃(CH₂)₉ | O | | 57 | |
| 8b | CH₃(CH₃)₁₄ | O | | 82 | 750 |
| 8c | CH₃(CH₂)₁₄ | H₂OH | | 50 | 1000 |
| com- | | | | 100 | 1 |
| pactin | | | | | |

^{a}IC₅₀ is the concentration required to inhibit HMG-CoA reductase by 50% of control.

As used herein, the term “HMG-CoA” is used interchangeably with “3-hydroxy-3-methylglutaryl-coenzyme A” and refers to an intermediate in the mevalonate pathway. HMG-CoA has a structure as shown in FIG. 2 and is formed from acetyl-CoA and acetoacetyl-CoA by HMG-CoA synthase. HMG-CoA can be converted by HMG-CoA reductase into mevalonic acid.

As used herein, the term “HMGR” is used interchangeably with “HMG-CoA reductase” and refers to the enzyme that converts HMG-CoA to mevalonate. The reaction catalyzed by HMG-CoA reductase is the rate limiting step of cholesterol biosynthesis.

As used herein, the term “hypercholesterolemia” refers to the presence of high levels of cholesterol in the blood. Hypercholesterolemia is a metabolic derangement that can be secondary to many diseases; it can also contribute to many diseases, such as, for example, cardiovascular diseases. In some embodiments, hypercholesterolemia is defined clinically by one or both of a) a total cholesterol level of greater than or equal to 240 mg/dL and b) an LDL (low density lipoprotein) cholesterol level of greater than or equal to 160 mg/dL.

As used herein, the term “hyperlipidemia” refers to the presence of excess lipids and/or lipoproteins in the blood. Excess lipids and lipoproteins that may be present in a hyperlipidemic subject include, without limitation, LDL cholesterol, HDL cholesterol, triglycerides, apolipoproteins, and cholesterol remnants. A hyperlipidemic subject may be both hypercholesterolemic and hypertriglyceridemic. In some embodiments, hypertriglyceridemia is characterized by a triglyceride level of greater than or equal to 200 mg/dL. In some embodiments, hypertriglyceridemia is characterized by a triglyceride level of greater than or equal to 250 mg/dL. In some embodiments, hypertriglyceridemia is characterized by a triglyceride level of greater than or equal to 400 mg/dL. In some embodiments, hyperlipidemia is defined clinically by one or more of the following thresholds in addition to meeting the threshold for hypertriglyceridemia: a) a total cholesterol level of greater than or equal to 240 mg/dL; b) an LDL (low density lipoprotein) cholesterol level of greater than or equal to 160 mg/dL; and c) an HDL (high density lipoprotein) cholesterol level of less than or equal to 40 mg/dL.

As used herein, the term “immunomodulation” refers to the immediate or delayed enhancement or reduction of the activity of at least one pathway involved in an immune response, whether this response is naturally occurring or artificially triggered, whether this response takes place as part of innate immune system or adaptive immune system or the both. In some embodiments, reduction of at least one pathway involved in the immune system leads to suppression of an immune response.

As used herein, the term “immunomodulator” refers to an agent whose action on the immune system leads to the immediate or delayed enhancement or reduction of the activity of at least one pathway involved in an immune response, whether this response is naturally occurring or artificially triggered, whether this response takes place as part of innate immune system or adaptive immune system or the both. An MHC Class II-mediated immunomodulator is an immunomodulator whose key action on the immune system involves molecules of MHC class II.

As used herein, the term “immunosuppressor” refers to an agent whose action on the immune system leads to the immediate or delayed reduction of the activity of at least one pathway involved in an immune response, whether this response is naturally occurring or artificially triggered, whether this response takes place as part of innate immune system or adaptive immune system or the both. An MHC Class II-mediated immunosuppressor is an immunosuppressor whose key action on the immune system involves molecules of MHC class II.

The terms “individual” and “subject” are used herein interchangeably. They refer to a human or another mammal (e.g., mouse, rat, rabbit, dog, cat, cattle, swine, sheep, horse or primate) that can be afflicted with or is susceptible to a disease or disorder (e.g., cancer, macular degeneration, etc.) but may or may not have the disease or disorder. In many embodiments, the subject is a human being. In many embodiments, the subject is a patient. Unless otherwise stated, the terms “individual” and “subject” do not denote a particular age, and thus encompass adults, children, and newborns.

As used herein, the term “inhibit” means to prevent something from happening, to delay occurrence of something happening, and/or to reduce the extent or likelihood of something happening.

As used herein, the phrase “islet of Langerhans cells” refers to a heterogeneous populations of cells that comprise one or more of glucagon-secreting alpha-cells, insulin-secreting beta-cells, somatostatin-secreting delta-cells, endothelial cells, and resident immune cells. Beta-cells can secrete insulin in a glucose regulated manner. In some embodiments, islet of Langerhans cells are transplanted into patients with insulin-related disorders, such as Type 1 Diabetes Mellitus (T1DM).

As used herein, the phrase “metabolic syndrome” refers to a syndrome characterized by a group of metabolic risk factors in one person. Such risk factors include abdominal obesity (excessive fat tissue in and around the abdomen), atherogenic dyslipidemia (blood fat disorders such as, for example, high triglycerides, low HDL cholesterol and high LDL cholesterol, that foster plaque buildups in artery walls), elevated blood pressure, insulin resistance or glucose intolerance, prothrombotic state (e.g., high fibrinogen or plasminogen activator inhibitor-1 in the blood) and proinflammatory state (e.g., elevated C-reactive protein in the blood). Presence of a few or all of these risk factors may constitute diagnosis for metabolic syndrome. Subjects with the metabolic syndrome are at increased risk of coronary heart disease and other diseases related to plaque buildups in artery walls.

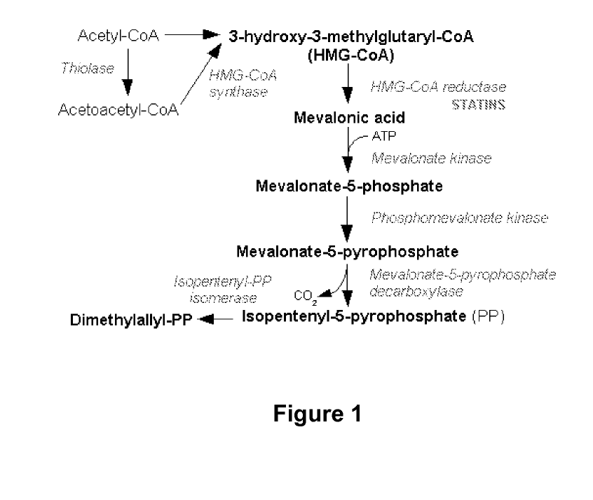

As used herein, the phrase “mevalonate pathway” is used interchangeably with “HMG-CoA reductase pathway” and refers to a metabolic pathway that produces, among other things, bile acids quinones, and steroids, including cholesterol and other isoprenoids. A schematic depicting the mevalonate pathway is shown in FIG. 1. The mevalonate pathway is also known as the isoprenoid pathway.

As used herein, the phrase “mevalonate pathway secondary metabolite” refers to a metabolite that is produced secondarily to inhibition or suppression of one or more components in the mevalonate pathway. In some embodiments, the secondary metabolite is produced secondarily to the inhibition of HMG-CoA reductase. For example, the secondary metabolite may be HMG, 2-hydroxyglutarate, 2-oxoglutarate, or a combination thereof. Alternatively or additionally, the secondary metabolite may be one that is identified in screening methods of the invention as discussed herein. In some embodiments, the secondary metabolite is a normal component of the mevalonate pathway but is produced at non-physiological levels in response to inhibition or genetic suppression of one or more components in the mevalonate pathway. For example, the secondary metabolite may be mevalonate expressed at supraphysiological levels.

As used herein, the phrase “mevalonate pathway secondary metabolite agent” refers to a secondary metabolite of the mevalonate pathway and structurally related compounds (e.g., analogs, derivatives, etc.). In some embodiments, the mevalonate pathway secondary metabolite agent is an HMG (3-hydroxy-3-methylglutaric acid) agent. In some embodiments, mevalonate pathway secondary metabolite agents inhibit HMG-CoA reductase. In some embodiments, mevalonate pathway secondary metabolite agents mediate one or more pleiotropic effects of statins.

As used herein, the term “mevalonic acid” (also known as 3,5-dihydroxy-3-methylpentanoic acid) refers to a compound having the structure depicted in FIG. 2 and is a precursor in the mevalonate pathway. As used herein, “mevalonate” refers to a salt of mevalonic acid.

As used herein, the term “neovasculature” refers to newly formed blood vessels that have not yet fully matured, i.e., do not have a fully formed endothelial lining with tight cellular junctions or a complete layer of surrounding smooth muscle cells. As used herein, the term “neovessel” is used to refer to a blood vessel in neovasculature.

The terms “normal” and “healthy” are used herein interchangeably. They refer to an individual or group of individuals who do not have a particular disease or condition. The term “normal” is also used herein to qualify a tissue sample isolated from a healthy individual.

The terms “pharmaceutical agent”, “therapeutic agent” and “drug” are used herein interchangeably. They refer to a substance, molecule, compound, agent, factor or composition effective in the treatment, inhibition, and/or detection of a disease, disorder, or clinical condition.

A “pharmaceutical composition” is herein defined as a composition that comprises an effective amount (or a unit dose within a therapeutically effective dosing regimen) of at least one active ingredient (e.g., a 3-hydroxy-3-methylglutaric acid agent), and at least one pharmaceutically acceptable carrier.

As used herein, the term “pharmaceutically acceptable carrier” refers to a carrier medium which does not interfere with the effectiveness of the biological activity of the active ingredient(s) and which is not excessively toxic to the host at the concentration at which it is administered. The term includes solvents, dispersion media, coatings, antibacterial and antifungal agents, isotonic agents, absorption delaying agents, and the like. The use of such media and agents for pharmaceutically active substances is well known in the art (see for example, “Remington's Pharmaceutical Sciences”, E. W. Martin, 18^{th} Ed., 1990, Mack Publishing Co.: Easton, Pa., which is incorporated herein by reference in its entirety).

As used herein, the phrases “pleiotropic effect mediated by statins,” and “pleiotropic effect of statins” refer to a beneficial effect mediated by statins that is not directly related to cholesterol. Pleiotropic effects of statins include, without limitation, pro-angiogenic, pro-vasculogenic, pro-vascular function, anti-inflammatory, anti-septic and immunomodulatory activities. For example, such activities may include, without limitation, one or more of improving endothelial cell function, activating stem or progenitor cells (e.g., by affecting mobilization, proliferation, differentiation, etc.), reducing inflammatory mediators and markers, improving microcirculation, stabilizing atherosclerotic plaques, reducing platelet and leukocyte adhesion, reducing thrombosis, modulating peroxisome proliferator-activated receptor function, reducing blood pressure, promotion of blood vessel formation, increased blood flow, vasodilation, and nitric oxide production.

As used herein, the term “preventing” when used to refer to the action of an agent to a process (e.g., angiogenesis) means reducing extent of and/or delaying onset of such a process when the agent (e.g., a therapeutic agent) is administered prior to development of one or more symptoms or attributes associated with the process. The term “preventing” does not require absolute abolishment of the process.

As used herein, the term “progenitor cell” refers to unipotent or multipotent, committed or determined cells derived from stem cells. Progenitor cells undergo further differentiation and commitment to give rise to morphologically identifiable immature cells. Progenitor cells which may be used in accordance with the present invention include, but are not limited to, hematopoietic, neural, mesenchymal, gastrointestinal, muscle, cardiac muscle, kidney, skin, lung, and embryonic progenitor cells. Progenitor cells may be identified morphologically, kinetically, or operationally as further described below in the definition of a stem cell.

The terms “protein”, “polypeptide”, and “peptide” are used herein interchangeably, and refer to amino acid polymers of at least three amino acids and can be as many as several thousand amino acids, either in their neutral (uncharged) forms or as salts, and either unmodified or modified by glycosylation, acylation, isoprenylation, side chain oxidation, or phosphorylation. In certain embodiments, the amino acid polymer is a full-length native protein. In other embodiments, the amino acid polymer is a smaller fragment of a full-length protein. In still other embodiments, the amino acid polymer has a sequence modified as compared with a native protein by additional substituents attached to the amino acid side chains, such as glycosyl units, lipids, or inorganic ions such as phosphates, as well as modifications relating to chemical conversion of the chains, such as oxidation of sulfhydryl groups. Thus, the term “protein” (or its equivalent terms) is intended to include amino acid polymers whose sequence is identical to that of full-length native protein, and also polymers whose amino acid sequence includes modifications that do not change specific properties of the polymer. In particular, the term “protein” encompasses protein isoforms, i.e., variants that are encoded by the same gene, but that differ in their pI or MW, or both. Such isoforms can differ in their amino acid sequence (e.g., as a result of alternative slicing or limited proteolysis), or in the alternative, may arise from differential post-translational modification (e.g., glycosylation, acylation or phosphorylation).

As used herein, the phrase “reperfusion injury” refers to damage to tissue caused when blood supply returns to the tissue after a period of ischemia. Absence of oxygen and nutrients from blood creates a condition in which restoration of circulation results in inflammation and oxidative damage through the induction of oxidative stress rather than restoration of normal function.

As used herein, the phrases “secondary metabolite,” “alternate metabolite”, and “alternative metabolite”, refer to organic compounds that are produced secondarily to inhibition, suppression, or modulation of a compound and/or enzyme in a particular metabolic pathway. In some embodiments, the secondary metabolite is a small molecule.

As used herein, the term “small molecule” includes any chemical or other moiety whose molecular weight is less than about 5000 daltons (Da). In some embodiments, small molecules have molecular weights below about 2500, about 1000, or about 500 daltons. In some embodiments, small molecules are not polymers. In some embodiments, small molecules are not peptides. In some embodiments, small molecules are not nucleic acids. In some embodiments, small molecules have biological activity and/or act to affect biological processes. Small molecules can include any number of therapeutic agents presently known and used, or can be small molecules synthesized in a library of such molecules for the purpose of screening for biological function(s).

As used herein, the term “statin” refers to an inhibitor of HMG-CoA reductase. Statins can be, for example, synthetic, naturally occurring compounds, and/or fermentation-derived. Examples of statins include, without limitation, atorvastin (marketed as Lipitor and Torvast), cerivastatin (marketed as Lipobay and Baycol), compactin, fluvastatin (marketed as Lescol and Lescol XL), lovastatin (marketed as Mevacor, Altocor, and Altoprev), mevastatin, mevinolin, NK-104, pitavastatin (marketed as Livalo and Pitava), pravastatin (marketed as Pravachol, Selektine, and Lipostat), rosuvastatin (marketed as Crestor), simvastatin (marketed as Zocor and Lipex), and derivatives and analogs thereof.

As used herein, the term “stem cell” refers to any pluripotent cell that under the proper conditions will give rise to a more differentiated cell. Stem cells which may be used in accordance with the present invention include hematopoietic, neural, mesenchymal, gastrointestinal, muscle, cardiac muscle, kidney, skin, lung, and embryonic stem cells. To give but one example, a hematopoietic stem cell can give rise to differentiated blood cells (i.e., red blood cell (erythrocyte), white blood cell (T-cell, B-cell, neutrophil, basophil, eosinophil, monocyte, macrophage), or platelet) or neural or muscle cells. In terms of morphology, hematopoietic stem cells are small mononuclear cells normally found in the bone marrow of adults. These cells can be mobile and can also be found in the blood at a concentration of 1-5 per 10⁵ nucleated cells. During development, hematopoietic stem cells may be found in various locations in the body including the liver, spleen, thymus, lymph nodes, yolk sac, blood islands, and bone marrow.

Stem cells can also be characterized by their ability (1) to be self-renewing and (2) to give rise to further differentiated cells. This has been referred to as the kinetic definition.

An operational definition of stem cell regards the stem cell as a colony-forming unit in various laboratory systems. For example, when suspensions of bone marrow (i.e., hematopoietic) cells are injected intravenously into heavily irradiated mice in which the spleen and marrow are reduced to stroma and are hematologically empty, discrete macroscopic colonies of cells are observed in the animal's spleen after 8-10 days. A cell that meets any one of these three definitions of stem cell is considered to be a stem cell according to the present invention.

As used herein, the term “susceptible” means having an increased risk for and/or a propensity for (typically based on genetic predisposition, environmental factors, personal history, or combinations thereof) something, i.e., a disease, disorder, or condition such as an inflammatory condition, an autoimmune disorder, etc., than is observed in the general population. The term takes into account that an individual “susceptible” for a condition may never be diagnosed with the condition.

The term “tissue” is used herein in its broadest sense. A tissue may be any biological entity that can (but does not necessarily) comprise a tumor cell. In the context of the present invention, in vitro, in vivo and ex vivo tissues are considered. Thus, a tissue may be part of an individual or may be obtained from an individual (e.g., by biopsy). Tissues may also include sections of tissue such as frozen sections taken for histological purposes or archival samples with known diagnosis, treatment and/or outcome history. The term tissue also encompasses any material derived by processing the tissue sample. Derived materials include, but are not limited to, cells (or their progeny) isolated from the tissue. Processing of the tissue sample may involve one or more of: filtration, distillation, extraction, concentration, inactivation of interfering components, addition of reagents, and the like.

The term “treatment” is used herein to characterize a method or process that is aimed at (1) delaying or preventing the onset of a disease, disorder, or condition; (2) slowing down or stopping the progression, aggravation, or deterioration of one or more symptoms of the disease, disorder, or condition; (3) bringing about ameliorations of the symptoms of the disease, disorder, or condition; (4) reducing the severity or incidence of the disease, disorder, or condition; or (5) curing the disease, disorder, or condition. A treatment may be administered prior to the onset of the disease, disorder, or condition, for a prophylactic or preventive action. Alternatively or additionally, the treatment may be administered after initiation of the disease, disorder, or condition, for a therapeutic action.

As used herein, the phrase “vascular function” refers to the transport of blood to deliver oxygen, nutrients and chemicals to cells of the body to ensure their survival and proper function, to remove cellular wastes, and/or to deliver immune, platelet, progenitor, and other cells to sites where they are needed. In some embodiments, vascular function can be improved by increasing availability of nitric oxide, reducing inflammation, oxidation, or thrombosis, maintaining or repairing endothelial tissue, and/or inhibiting vascular smooth muscle proliferation and migration.

As used herein, the term “wound” refers to a type of injury in which the skin is torn, cut or punctured (an open wound), or where blunt force trauma causes a contusion (a closed wound).

### Detailed Description of Certain Embodiments

The present invention is directed, among other things, to using secondary metabolites in the mevalonate pathway (such as, for example, HMG) and/or structurally related compounds to mediate biological activities (e.g., for therapeutic applications) and/or as diagnostic agents. In some embodiments, the biological activities comprise one or more pleiotropic effects of statins (such as, for example, angiogenesis, promoting vascular function, anti-inflammatory action, immunomodulation, etc.). Also provided are methods of screening for mevalonate pathway secondary metabolites, methods of producing HMG, and methods of diagnosing comprising measuring amount of mevalonate pathway secondary metabolites.

In accordance with the present invention, there may be employed conventional molecular biology, microbiology, and recombinant DNA techniques within the skill of the art. Such techniques are explained fully in the literature. See, e.g., Maniatis, Fritsch & Sambrook, “Molecular Cloning: A Laboratory Manual”, 1982; “DNA Cloning: A Practical Approach,” Volumes I and II, D. N. Glover (Ed.), 1985; “Oligonucleotide Synthesis”, M. J. Gait (Ed.), 1984; “Nucleic Acid Hybridization”, B. D. Hames & S. J. Higgins (Eds.), 1985; “Transcription and Translation” B. D. Hames & S. J. Higgins (Eds.), 1984; “Animal Cell Culture”, R. I. Freshney (Ed.), 1986; “Immobilized Cells And Enzymes”, IRL Press, 1986; B. Perbal, “A Practical Guide To Molecular Cloning”, 1984.

I. Methods of Screening for Secondary Metabolites and Related Compounds with Beneficial Effects

In certain embodiments, the invention provides methods for screening for secondary metabolites in the mevalonate pathway that may have beneficial effects. Such methods generally comprise steps of (a) applying at least one inhibitor of at least one component in the mevalonate pathway to a system comprising cells and (b) identifying compounds whose amounts are higher in the absence of the inhibitor as compared to in its presence.

Mevalonate pathway inhibitors that can be used in accordance with inventive methods include, without limitation, inhibitors of HMG-CoA reductase, commonly known as statins. Statins can be, for example, synthetic, naturally occurring compounds, and/or fermentation-derived. Examples of statins include, without limitation, atorvastin (marketed as Lipitor and Torvast), cerivastatin (marketed as Lipobay and Baycol), compactin, fluvastatin (marketed as Lescol and Lescol XL), lovastatin (marketed as Mevacor, Altocor, and Altoprev), mevastatin, mevinolin, NK-104, pitavastatin (marketed as Livalo and Pitava), pravastatin (marketed as Pravachol, Selektine, and Lipostat), rosuvastatin (marketed as Crestor), simvastatin (marketed as Zocor and Lipex), and derivatives and analogs thereof. Inhibitors and/or derivatives thereof can be used in combination with each other and/or other agents.

Other enzymes that act in the mevalonate pathway include farnesyl transferase and geranylgeranyl transferase, which transfer farnesyl and geranylgeranyl moieties respectively to proteins. These hydrophobic moieties are often embedded into cellular membranes and serve to anchor otherwise hydrophilic proteins to membranes. Inhibitors of farnesyl transferase and geranylgeranyl transferase may also be suitable for use in inventive methods. Suitable inhibitors include, for example, any of a variety of farnesyl transferase and geranylgeranyl transferase inhibitors, including some that are in development. Because many of the small G proteins that are isoprenylated by these enzymes are involved in regulating the growth and proliferation pathways, several farnesyl transferase and geranylgeranyl transferase inhibitors are being developed as anti-cancer agents. Non-limiting examples of farnesyl transferase inhibitors include tetrapeptidic pseudosubstrates (e.g., CAAX peptidomimetics such as, for example, the CAAX tetrapeptide (Cys-Val-Phe-Met); A-170634; AZD3409; BIM-46228; FTI-205; FTI-276; FTI-277; B-956; B-1086; BMS-193269; BZA2B; BZA5B; L-731,734; L-731,735; L-739,749; L-739,750; L-744,832; L-745,631; L-778,123; TR062); farnesylgroup-containing inhibitors including farnesyl pyrophosphate analogs (such as, for example, alpha-Hydroxyfarnesylphosphate, farnesylmethylhydroxyphosphinyl methyl phosphonic acid, TR006, and TR015), bisubstrate analogs (such as, for example, BMS-186511, BMS-184878), naturally occurring inhibitors (such as, for example, manumycin), and others (such as, for example, tipifarnib (Zarnestra™ R115777), lonafarnib (SCH66336), PD083176, BMS-214662, RPR 130401, and SCH-44342). Non-limiting examples of geranylgeranyl transferase inhibitors include CAAL peptidomimetics, GGTI-286, GGTI-298, GGTI-2154, GGTI-2166, GGTI-2418, and TR031.

Squalene synthase inhibitors act at a late step in the mevalonate pathway and may block the production of sterols without interfering with the production of many other isoprenoids. Accordingly, squalene synthase inhibitors may be used in the practice of the present invention. A variety of squalene synthase inhibitors are known in the art and may be suitable for use. Non-limiting examples of squalene synthase inhibitors include lapaquistat, zaragozic acids (such as zaragozic acids A, B, and C), BMS-18774, BMS-188494 (the ester prodrug of BMS-18774) E5700, EP2302, EP2306, ER119884, ER27856 and other lipophilic 1,1-bisphosphonates, RPR-107393, TAK-475, and YM-53601.

Alternatively or additionally, mevalonate pathway secondary metabolites can be obtained from biological material and/or individuals that harbor genetic deficiencies affecting the mevalonate pathway. For example, Hyperimmunoglobulin D syndrome (HIDS) and mevalonate kinase (MVK) deficiency are two related genetic disorders that are distinguished by the severity of symptoms. HIDS, the milder of the two, occurs in patients who have a moderate loss of MVK activity and is characterized by an elevation of type D immunoglobulins and recurrent bouts of fever, joint pain, and skin rash (Houten et al (2003)). MVK deficiency results from a near complete loss of MVK activity and is characterized by recurrent bouts of fever, mental retardation, and ataxia (Prietsch et al. (2003)). Elevated levels of mevalonic acid are present in the blood of patients of HIDS and MVK deficiency as a result of increased flux through HMG-CoA reductase to compensate for the reduced level of metabolites downstream of MVK.

Systems to which inhibitors can be applied include, without limitation, cell culture systems and living organisms. For example, cells can be obtained from tissues, frozen stocks, other cell cultures, etc. and grown in cell culture medium. Living organisms such as laboratory animals, plants, fungi, etc. may also or alternatively be used. Systems are generally treated with the inhibitor and a sample collected from such systems for analysis. Generally, systems used in inventive methods comprise cells that express enzymes that act in the mevalonate pathway, such as HMG-CoA reductase. In some embodiments, the cells naturally express enzymes acting in the mevalonate pathway. For example, hepatocytes, endothelial cells, and immune cells naturally express HMG-CoA reductase and can be used in accordance with the invention. In some embodiments, the cells are engineered to express enzymes such as HMG-CoA reductase at levels higher than they normally express such enzymes. In some such embodiments, the cells do not endogenously express HMG-CoA reductase or express HMG-CoA reductase at low levels.

The step of identifying generally comprises performing one or more techniques that allow identification and quantification of compounds in a given sample, such as gas chromatography-mass spectrometry (GC-MS), high performance liquid chromatography (HPLC), liquid chromatography-mass spectrometry, gas-liquid chromatography, mass spectrometry (MS), ion chromatography, and enzyme-linked immunosorbent assay (ELISA). Compounds whose amounts have increased after and/or as a result of application of the inhibitor to the system are identified. To determine that the amount of a compound is increased, any of variety of comparisons may be made. For example, the amount of a given compound after application of the inhibitor to the system may be compared to the amount in a sample from the same system before application of the inhibitor to the system. Alternatively or additionally, the amount of a given compound after application of the inhibitor to the system (the “test” system) may be compared to the amount in a sample from a control system. The control system may, for example, be run in parallel with the “test” system such that all or most conditions for the two systems are the same, except that the control system is not treated with an inhibitor. The control system may not be treated with any agent, or may be treated with an agent that is known not to inhibit a component of the mevalonate pathway. An amount of a compound from a sample after application of the inhibitor may be compared to a reference amount, wherein the reference amount represents an expected amount of the compound in the absence of the inhibitor. The reference amount may be obtained from any of a variety of sources, such as, for example, published literature, archived data from other experiments, etc.

In some embodiments, methods further comprise a step of testing compounds identified in step (b) and/or a structurally related compound (e.g, an analog, derivative, etc.) for a biological activity. In some embodiments, compounds are characterized for toxicity, efficacy, and/or potency in mediating one or more biological activities. It is a contemplation of this invention that compounds other than those identified by screening methods described above may also be characterized and/or tested for biological activity. For example, mevalonate pathway secondary metabolites that are identified by other methods, or known in the art, and/or their related compounds may also be tested and/or characterized for biological activity. In some embodiments, HMG and/or a structurally related compound of HMG (herein referred to as “HMG agent”) is characterized and/or tested for biological activity.

In some embodiments, the biological activity comprises at least one pleiotropic effect of statins such as those described herein. Non-limiting examples of pleiotropic effects include, without limitation, pro-angiogenic, pro-vasculogenic, pro-vascular function, anti-inflammatory, anti-septic, and immunomodulatory activities. Non-limiting examples of such activities include, without limitation, one or more of improving endothelial cell function, activating stem or progenitor cells (e.g., by affecting mobilization, proliferation, differentiation, etc.), reducing inflammatory mediators and markers, improving microcirculation, stabilizing atherosclerotic plaques, reducing platelet and leukocyte adhesion, reducing thrombosis, modulating peroxisome proliferator-activated receptor function, reducing blood pressure, promotion of blood vessel formation, increased blood flow, vasodilation, and nitric oxide production.

A compound may be tested for its ability to induce one or more of any of a variety of biological activities as discussed above. Examples 1-4 provide non-limiting examples of tests that can be used to assess pro-angiogenic and pro-vascular function effects. In Examples 1-4, a compound, HMG, is tested for its ability to promote angiogenesis, improve vascular function (as measured by increased blood flow), induce endothelial cell function, induce endothelial tube formation, and induce endothelial cell migration.

Alternatively or additionally, other tests for similar and/or other biological activities may be used. For example, to test compounds for their anti-inflammatory activities, cells can be subjected to an inflammatory stimulus (such as, for example, lipopolysaccharide) in the presence or absence of the compound, then inflammation response indicators (such as, for example, biomarkers (e.g, C reactive protein), mediators (e.g., inducible nitric oxide synthase (iNOS)), and/or signaling components mediators (e.g., NF-kappa B) can be measured. To evaluate involvement of peroxisome proliferator-activated receptors (PPARs) in mediating anti-inflammatory effects of mevalonate pathway secondary metabolites, cells in which one or more PPAR isoforms (e.g., alpha, gamma, delta) has/have been genetically suppressed can be exposed an inflammatory stimulus and to one or more anti-inflammatory compounds, and the responses of such cells compared to that of normal (i.e., not suppressed in one or more PPAR isoforms) cells.

### II. Methods of Producing HMG

As discussed herein, the inventors have discovered that HMG has biological activity associated with pleiotropic effects of statins and may mediate such effects. For example, HMG has pro-angiogenic effects and promotes vascular function. HMG therefore has therapeutic value with a potentially broad range of therapeutic applications.

In certain embodiments, the invention provides methods of producing HMG comprising steps of applying at least one HMG-CoA reductase inhibitor to a system comprising cells and obtaining 3-hydroxy-3-methylglutaric acid (HMG) from the system.

HMG-CoA reductase inhibitors that can be used in accordance with inventive methods include, without limitation, statins as described herein. Systems may comprise cell culture systems and/or living organisms as described herein. For example, cell cultures comprising cells that express HMG-CoA reductase may be treated with one more HMG-CoA reductase inhibitors and HMG may be subsequently harvested from the cell culture medium and/or cells. Similarly, a living organism that comprises cells that express HMG-CoA reductase may be treated with HMG-CoA, and HMG subsequently collected from biological material (e.g., tissues, bodily fluids, etc.) from the organism.

A variety of methods to quantify and/or purify 3-hydroxy-3-methylglutaric acid (HMG) are known in the art and include, without limitation, gas chromatography/mass spectrometry (GC/MS), thin layer chromatography, paper chromatography, and liquid chromatography/mass spectrometry (LC/MS). Sample collection, extraction, and preparation methods appropriate for measuring organic acids are known in the art. For example, levels of HMG produced by cultured cells or in biological fluids may be measured before and/or after administration of an HMG-CoA reductase inhibitor by GC/MS. Tissue and/or biological fluids are extracted with a sodium chloride and hydrochloric acid extraction solvent, products are derivatized with N,O-bis-trimethylsilyltrifluoroacetamide, and products are analyzed by GC/MS as described by Jones M G and Chalmers R A (2000), the entire contents of which are herein incorporated by reference.

### III. Methods of Mediating Biological Activities

In certain embodiments, the invention provides methods of mediating one or more biological activities using a mevalonate pathway secondary metabolite agent. In some embodiments, the agent is a 3-hydroxy-3-methylglutaric acid agent. In some embodiments, the biological activity comprises at least one pleiotropic effect of statins such as those described herein. Non-limiting examples of pleiotropic effects include, without limitation, pro-angiogenic, pro-vasculogenic, pro-vascular function, anti-inflammatory, and immunomodulatory activities. Non-limiting examples of such activities include, without limitation, one or more of improving endothelial cell function, reducing inflammatory mediators and markers, improving microcirculation, stabilizing atherosclerotic plaques, reducing platelet and leukocyte adhesion, reducing thrombosis, reducing blood pressure, promotion of blood vessel formation, increased blood flow, vasodilation, and nitric oxide production.

In certain embodiments, the invention provides methods of promoting blood vessel formation and/or vascular function comprising a step of administering a mevalonate pathway secondary metabolite agent to a subject in an amount effective such that extent of blood vessel formation or vascular function is increased as compared with that observed or expected for a control subject to whom a mevalonate pathway secondary metabolite agent is not administered.

In certain embodiments, the invention provides methods comprising a step of administering to a subject suffering from or susceptible to an inflammatory disease or condition, a condition for which immunomodulation is desirable, or both a mevalonate pathway secondary metabolite agent in an amount effective to ameliorate the disease or condition and/or modulate the immune system.

In certain embodiments, the invention provides methods useful for enhancing cell-based therapies, comprising administering to cells a mevalonate pathway secondary metabolite agent and administering such cells to a subject in need of cell therapy.

In some embodiments, the mevalonate pathway secondary metabolite agent enhances, mobilization, migration, and/or homing of cells used for cell therapy. In some embodiments, the mevalonate pathway secondary metabolite agent enhances proliferation and/or differentiation of the cells. In some embodiments, the mevalonate pathway secondary metabolite agent enhances engraftment and/or revascularization of the cells. In some embodiments, the mevalonate pathway secondary metabolite agent prevents or reduces host immune-inflammatory responses to the cells.

In some embodiments, the cells comprise stem cells and/or progenitor cells. For stem cells and/or progenitor cells to affect tissue repair or regeneration at a distant wound site, these cells are generally mobilized from their site of residence. They then migrate and/or home to the site of wounding or tissue damage, proliferate, and differentiate into the cell type(s) they are replacing at the site of damage. Treating stem cells and/or progenitor cells in vitro with a mevalonate pathway secondary metabolite agent can prime the cells in a manner that enhances their mobilization, migration, homing, proliferation, and/or differentiation when they are administered to a patient in need of tissue repair or regeneration.

In some embodiments, the cells comprise islet of Langerhans cells. Islets of Langerhans are heterogeneous populations of cells that comprise glucagon-secreting alpha-cells, insulin-secreting beta-cells, somatostatin-secreting delta-cells, endothelial cells, and resident immune cells. Fully differentiated islets from adult donors have been transplanted into patients with Type 1 Diabetes Mellitus (T1DM) to help these patients achieve independence from exogenous insulin and to reduce the secondary complications arising from T1DM. Beta-cells, through their ability to secrete insulin in a glucose regulated manner, are a major source of the beneficial effects of transplanted islets. The number of donor islets is limiting and adult beta-cells proliferate at a very low rate. Thus, methods of expanding the number of beta-cells available for transplantation are desirable. Likewise, for islet transplants to function successfully, these engrafted islets need to form new vasculature to receive nutrients, monitor blood glucose levels, and transport insulin released by the transplanted islet beta-cells. Thus, agents that promote revascularization of transplanted islets are beneficial to the function of such transplanted islets. Furthermore, for lasting independence from exogenous insulin to be achieved, the host immune rejection response to the transplanted islets should be prevented. Agents that minimize or prevent such host immune rejection responses are desirable. In some embodiments, islets are treated with a mevalonate pathway secondary metabolite agent. In some such embodiments, islets are treated in vitro prior to transplantation into T1DM patients to enhance beta-cell proliferation, prime islet endothelial cells for revascularization, and/or to reduce host immune rejection responses.

### A. Mevalonate Pathway Secondary Metabolite Agents

As used herein, the phrase “mevalonate pathway secondary metabolite agent” refers to a secondary metabolite of the mevalonate pathway and structurally related compounds (e.g., analogs, derivatives, etc.). In some embodiments, the mevalonate pathway secondary metabolite agent is an HMG (3-hydroxy-3-methylglutaric acid) agent. In some embodiments, mevalonate pathway secondary metabolite agents inhibit HMG CoA reductase. In some embodiments, mevalonate pathway secondary metabolite agents mediate one or more pleiotropic effects of statins.

As used herein, the term “HMG agent” is used interchangeably with “3-hydroxy-3-methylglutaric acid agent” and refers to 3-hydroxy-3-methylglutaric acid (HMG) and structurally related compounds (e.g., analogs, derivatives, etc.). In some embodiments, such structurally related compounds include alkyl derivatives such as 3-alkyl-3-hydroxyglutaric acids. (See, for example, Baran et al. 1985. “3-Alkyl-3-hydroxyglutaric Acids: A New Class of Hypocholesterolemic HMG CoA Reductase Inhibitors.” J. Med. Chem. 28:597-601, the contents of which are hereby incorporated by reference in their entirety.) Examples of 3-alkyl-3-hydroxyglutaric acids include, without limitation, 3-n-pentadecyl-3-hydroxyglutaric acid, and other compounds having core structures depicted in FIG. 4 and whose substituent groups are listed in Table 1.

TABLE 1
HMGR inhibition by 3-alkyl-3-hydroxyglutaric acids
and their derivatives (from Baran et al.)

| | | | | in vitro inhibition | |
|---|---|---|---|---|---|
| | | | | of HMGR | |
| com- | | | | | IC₅₀^{a} |
| pound | R | X | Y | @10⁻³ M | (μM) |
| 3a | CH₃ | | | | |
| 3b | CH₃CH₂ | | | 29 | |
| 3c | CH₃(CH₂)₂ | | | 37 | |
| 3d | (CH₃)₂CH | | | 10 | |
| 3e | CH₃(CH₂)₅ | | | 5 | |
| 3f | CH₃(CH₂)₉ | | | 86 | 100 |
| 3g | CH₃(CH₂)₁₂ | | | 97 | 50 |
| 3h | CH₃(CH₂)₁₃ | | | 95 | 130 |
| 3j | CH₃(CH₂)₁₄ | | | 99 | 50 |
| 3k | CH₃(CH₂)₁₅ | | | 90 | 100 |
| 3l | CH₃(CH₂)₁₆ | | | 85 | 100 |
| 3m | CH₃(CH₂)₁₈ | | | 86 | 350 |
| 4a | CH₃ | OC₂H₅ | OC₂H₅ | 0 | |
| 4b | CH₃ | OCH₃ | NH₂ | 0 | |
| 4c | CH₃(CH2)₉ | OC₂H₅ | OC₂H₅ | 0 | |
| 4d | CH₃(CH₂)₁₄ | OCH₃ | OCH₃ | −1 | |
| 4e | CH₃(CH₂)₁₄ | OC₂H₅ | OC₂H₅ | 36 | >1000 |
| 4f | CH₃(CH2)₁₄ | OH | OC₂H₅ | 81 | 900 |
| 4g | CH₃(CH2)₁₄ | OH | O(CH₂)₂N(CH₃)₂ | 0 | |
| 5a | CH₃ | O | | 0 | |
| 5b | CH₃ | NH | | 50 | |
| 5c | CH₃(CH₂)₁₄ | O | | 80 | 250 |
| 5d | CH₃(CH₂)₁₄ | O | COCH₃ | −9 | |
| 6 | CH₃(CH₂)₁₄ | | | 0 | |
| 7a | CH₃(CH₂)₉ | | | 0 | |
| 7b | CH₃(CH₂)₁₄ | | | 0 | |
| 8a | CH₃(CH₂)₉ | O | | 57 | |
| 8b | CH₃(CH₃)₁₄ | O | | 82 | 750 |
| 8c | CH₃(CH₂)₁₄ | H₂OH | | 50 | 1000 |
| com- | | | | 100 | 1 |
| pactin | | | | | |

^{a}IC₅₀ is the concentration required to inhibit HMG CoA reductase by 50% of control.

In some embodiments, the mevalonate pathway secondary metabolite agent is one that has been characterized in the art. In some embodiments, the mevalonate pathway secondary metabolite agent is a mevalonate pathway secondary metabolite identified using inventive methods described herein. In some embodiments, the mevalonate pathway secondary metabolite agent is a structurally related compound (e.g., analog, derivative, etc.) of a mevalonate pathway secondary metabolite that has been characterized and/or tested for biological activity.

### B. Blood Vessel Formation and Vascular Function

In some embodiments, the subject to whom mevalonate pathway secondary metabolite agent is administered suffers from a disease or condition that makes it desirable to promote angiogenesis and/or improve vascular function in the subject. In general, any subject having a disease or condition characterized by insufficient vascularization, and/or diminished vascular function may be administered mevalonate pathway secondary metabolite agent in accordance with the invention. Subjects having a predisposition to diseases or conditions characterized by insufficient vascularization and/or diminished vascular function may also be administered mevalonate pathway secondary metabolite agent in accordance with the invention.

For example, the subject may suffer from a disease or condition selected from the group consisting of hypertension, diabetic peripheral vascular disease (which may or may not involve having diabetic ulcers), gangrene, Buerger's syndrome, ischemia, occlusive vascular disease, obstructive vascular disease, peripheral vascular disease, myocardial infarction, coronary artery disease, visceral vascular disease, and combinations thereof. Examples of ischemias include, without limitation, cerebrovascular ischemia, renal ischemia, pulmonary ischemia, limb ischemia, ischemic cardiomyopathy, myocardial ischemia, and ischemia of particular tissues (such as, for example, muscle, brain, kidney and lung).

In some embodiments, the subject has a wound. In some such embodiments, the wound is a surgical wound. Administering mevalonate pathway secondary metabolite agent to such subjects may facilitate healing by promoting neovascularization and/or vascular function.

Blood vessel formation may comprise angiogenesis (generation of new blood vessels from existing endothelial cells) and/or vasculogenesis (generation of new blood vessels from stem cells)

A number of tests can be used in accordance with the invention to determine extent of blood vessel formation. A variety of such methods are well known in the art and are described, for example, in Harrison's: Principles of Internal Medicine (McGraw Hill, Inc., New York). Tests for blood vessel formation include tests that fall under the following categories: (1) nonspecific indexes of tissue necrosis and inflammation in a biopsy specimen, (2) electrocardiograms, (3) serum enzyme changes (e.g., creatine phosphokinase levels), and (4) blood vessel imaging. Blood vessel formation can be determined, for example, by limb blood pressure measurement, quantitative angiography, intravascular doppler blood flow measurements, evaluation of capillary density in a biopsy specimen, etc.

Any of a variety of aspects of vascular function may be assayed to determine whether extent of vascular is increased. For example, blood flow, vasodilation, nitric oxide production, blood oxygen saturation, peripheral circulation, thrombosis, platelet function, progenitor cell recruitment and development, immune cell recruitment and response, and/or function of signal transduction pathways such as the PI3K/Akt/eNOS axis, etc. may be assayed.

Extent of blood vessel formation and/or of vascular function may be determined in comparison to a control. As is understood by those of ordinary skill in the art, control values may be obtained and/or estimated in a variety of ways. Data from control subjects that are similar to the subject being administered may be used for comparisons. Additionally or alternatively, by collecting data at various time points during a treatment regimen, subjects may serve as their own longitudinal controls. Alternatively or additionally, standard values may be used as control values. Such standard values may be calculated and/or extrapolated from known data, values, parameters, etc., which may be available, for example, in clinical records, archives, literature, etc.

C. Reducing Inflammation and/or Immunomodulation

In some embodiments, the subject to whom mevalonate pathway secondary metabolite agent is administered suffers from a disease or condition that makes it desirable to reduce inflammation and/or modulate the immune system in the subject. Subjects to whom effective amounts of mevalonate pathway secondary metabolite agent can be administered include those suffering from or predisposed to an inflammatory disease or condition and/or a condition for which immunomodulation is desired. Inflammatory diseases and conditions include both acute and chronic inflammatory diseases and conditions. Many inflammatory diseases and conditions involve immune responses; likewise, many disorders of the immune system are characterized by an inflammatory condition. Thus, many of the below-mentioned diseases fall under both categories and are herein loosely termed “immuno-inflammatory diseases and conditions.”

Non-limiting examples of immuno-inflammatory diseases and conditions include acquired immune deficiency syndrome (AIDS), allograft rejection, adult respiratory distress syndrome, arthritis (including, for example, rheumatoid arthritis and osteoarthritis), asthma, atherosclerosis, autoimmune disorders (such as, for example. Addison's disease, autoimmune hepatitis Celiac disease, Crohn's Disease, giant cell arteritis, Goodpasture's syndrome, Grave's disease, Guillain-Barre syndrome, Hashimoto's thyroiditis, juvenile rheumatoid arthritis, lupus, polymyalgia rheumatica, psoriasis, primary biliary cirrhosis, rheumatoid arthritis, scleroderma, sclerosing cholangitis, Sjogren's syndrome, temporal arteritis, type 1 diabetes mellitus, ulcerative colitis, Wegener's granulomatosis, and combinations thereof), cancer, cerebral palsy, diabetes (including type 1 diabetes mellitus and type 2 diabetes mellitus), eczema, glomerulonephritis, heart failure, herpes dementia, immune complex diseases, infection caused by invasive microorganisms that produce nitric oxide (NO), inflammatory bowel disease, inflammatory sequelae of viral infections, ischemia (including ischemic brain edema), metabolic syndrome, migraine, multiple sclerosis, myocarditis, organ transplant/bypass disorders, osteoporosis, oxidant induced lung injury, Paget's disease, hyperimmunoglobulin D syndrome, mevalonate kinase deficiency, pain, peritonitis, reperfusion injury, retinitis, sepsis and/or septic shock, sickle cell anemia, stroke, toxic shock syndrome, uveitis, X-adenoleukodystrophy (X-ALD), and combinations thereof. (It is noted that some of ordinary skill in the art would classify multiple sclerosis as an autoimmune disorder and others would not.) Many neurodegenerative diseases are also characterized by an inflammatory condition. Such neurodegenerative diseases include, for example, Alzheimer's disease, Parkinson's disease, Landry-Guillain-Barre-Strohl syndrome, multiple sclerosis, viral encephalitis, acquired immunodeficiency disease (AIDS)-related dementia, amyotrophic lateral sclerosis, brain trauma, and spinal cord disorders.

In some embodiments, an immuno-inflammatory disease or condition is mediated by CD40. In some such embodiments, a mevalonate pathway secondary metabolite agent is administered in an amount effective to modulate CD40 expression. The CD40 expression that is modulated may comprise CD40 expression induced by IFNγ.

In some embodiments, a mevalonate pathway secondary metabolite agent is administered in an amount effective to alter the levels of one or more cytokines associated with inflammation and/or autoimmune disease. For example, the mevalonate pathway secondary metabolite agent may reduce the level of a cytokine that is typically elevated during inflammation and/or autoimmune disease. Alternatively or additionally, the mevalonate pathway secondary metabolite agent may increased the level of a cytokine that is typically decreased during inflammation and/or autoimmune disease. Examples of cytokines whose levels may be altered by an effective amount of a mevalonate pathway secondary metabolite agent include, but are not limited to, interleukin-1 (IL-1), interferon gamma (IFN γ), interleukin-17 (IL-17), interleukin-6 (IL-6), RANTES (also known as CCL5), and MCP-1 (also known as CCL2).

In some embodiments, a mevalonate pathway secondary metabolite agent is adminstered to a subject in whom immunomodulation is desired. Such subjects may or may not have one or more of the above-mentioned diseases and conditions. In some embodiments, immunomodulation comprises immunosuppression. For example, it may be desirable to induce immunosuppression in a subject undergoing organ or tissue transplantation. In such a subject, immunological incompatibilities are likely to exist between the subject receiving the graft and the graft donor. Cells of the recipient may detect the presence of non-self cells and are likely to kill cells being grafted, leading to rejection of the graft (i.e., allograft rejection). Improvement of graft tolerance can be achieved by immunosuppression.

Similarly, it may be desirable to induce immunosuppression in subjects receiving biological prostheses. Resilient, biocompatible two or more layered tissue prostheses can be engineered into a variety of shapes and used to repair, augment, or replace mammalian tissues and organs. Administering mevalonate pathway secondary metabolite agent according to methods of the invention may reduce or suppress inflammation and immune rejection at the site of implantation.

### D. Dosages

A mevalonate pathway secondary metabolite agent, or a pharmaceutical composition thereof, will generally be administered in such amounts (e.g., as a unit dose) and for such a time as is necessary or sufficient to achieve at least one desired result. For example, a mevalonate pathway secondary metabolite agent can be administered in such amounts and for such a time that it promotes angiogenesis, promotes vasculogenesis, promotes revascularization of undervascularized tissue, promotes wound healing, increases blood flow, increases nitric oxide production, induces endothelial cell proliferation, facilitates endothelial tube formation, induces endothelial cell migration, reduces levels of inflammatory cytokines, reduces or enhances T-cell proliferation, reduces or inhibits migration of leukocytes by chemotaxis, inhibits or reduces interferon gamma (IFN γ)-mediated expression of downstream genes, suppress rejection of a graft, and/or otherwise yields clinical benefits.

A dosing regimen according to the present invention may consist of a single dose or a plurality of doses over a period of time. Administration may be one or multiple times daily, weekly (or at some other multiple day interval) or on an intermittent schedule. For example, a dosing regimen may comprise administration of a single dose of chlorotoxin agent or administration of 2 doses, 3 doses, 4 doses, 5 doses, 6 doses or more than 6 doses. Two consecutive doses may be administered at 1 day interval, 2 days interval, 3 days interval, 4 days interval, 5 days interval, 6 days interval, 7 days interval, or more than 7 days interval (e.g., 10 days, 2 weeks, or more than 2 weeks). The exact amount of a 3-hydroxy-3-methylglutaric acid agent, or pharmaceutical composition thereof, to be administered will vary from subject to subject and may depend on several factors.

Depending on the route of administration, effective amounts may be calculated according to the body weight; body surface area; nature of disease or condition; tissues affected; etc. of the subject to be treated. Optimization of the appropriate dosages can readily be made by one skilled in the art in light of pharmacokinetic data observed in human clinical trials. The final dosage regimen may be determined by the attending physician or other clinician, considering various factors which modify the action of the drugs, e.g., the drug's specific activity, the severity of the damage and the responsiveness of the patient, the age, condition, body weight, sex and diet of the patient, the severity of any present infection, time of administration, the use (or not) of other therapies, and other clinical factors. As studies are conducted using 3-hydroxy-3-methylglutaric acid agents, further information will emerge regarding the appropriate dosage levels and duration of treatment.

Typical effective amounts comprise between about 0.01 mg/kg body weight to about 1000 mg/kg body weight. A therapeutically effective amount may vary from 0.01 mg/kg to about 1000 mg/kg. In some embodiments, effective amounts range from about 0.1 mg/kg to about 200 mg/kg and/or from about 0.2 mg/kg to about 20 mg/kg. In some embodiments, a therapeutically effective amount ranges from about 0.5 mg/kg/day to about 10 mg/kg/day.

### E. Routes of Administration and Pharmaceutical Compositions

Mevalonate pathway secondary metabolite agents, or pharmaceutical compositions thereof, may be administered using any administration route effective for achieving the desired therapeutic effect. The suitability of particular routes of administration may depend on the subject and/or the purpose. A route of administration selected may be selected depending on, for example, the particular agent being used, the severity of the condition being treated, the dosage required for therapeutic efficacy, etc. Methods of the invention may generally be practiced using any mode of administration that is medically acceptable, that is, any mode that produces effective levels of the active compounds without causing clinically unacceptable adverse effects. Routes of administration include, but are not limited to, topical (including epicutaneous, enema, eye drops, ear drops, intranasal, vaginal, etc.), enteral (including oral, feeding tube, rectal, etc.), parenteral (including intravenous, intraarterial, intramuscular, intracardiac, subcutaneous, intraosseous, intradermal, intrathecal, intraperitoneal, transdermal, transmucosal, inhalational, infusion etc.), epidural, and intravitreal.

In some embodiments, the mevalonate pathway secondary metabolite agent is delivered orally. Oral administration may be particularly suitable for subjects having a condition selected from the group consisting of hypertension, diabetes, gangrene, wounds, Buerger's syndrome, and other conditions characterized by reduction or obstruction in microvasculature, though any route of administration is possible. Oral administration may be particularly desirable for prophylactic treatment because of the convenience to the patient as well as the dosing schedule. Compositions suitable for oral administration may be presented as discrete units, such as capsules, tablets, lozenges, each containing a predetermined amount of the 3-hydroxy-3-methylglutaric acid agent. Other compositions include suspensions in aqueous liquids or non-aqueous liquids such as a syrup, elixir or an emulsion.

In some embodiments, the mevalonate pathway secondary metabolite agent is delivered locally to a tissue or to some tissues where a particular biological activity (e.g., angiogenesis, vasculogenesis, improved vascular function, reduce inflammation, immunomodulation, etc.) is desired. For example, local administration may be suitable for a subject having a condition that results from a severe occlusive and/or obstructive vascular tissue, mevalonate pathway secondary metabolite agent. Local administration may be achieved in some embodiments by inserting a device such as a stent, wherein the device comprises the 3-hydroxy-3-methylglutaric acid agent, into the tissue where angiogenesis and/or increased vascular function is desired. In some embodiments, local administration comprises administering to the subject a pharmaceutical composition containing a mevalonate pathway secondary metabolite agent and a pharmaceutically acceptable carrier. In some embodiments, the pharmaceutical composition is suitable for topical application or internal applications. In some such embodiments, the composition is formulated as a salve, a gel or a patch. The pharmaceutical composition may comprise a controlled release matrix and, optionally, the composition may be formulated to release the mevalonate pathway secondary metabolite agent substantially continuously for a period of at least one day. Exemplary controlled release formulations that may be suitable for delivering mevalonate pathway secondary metabolite agent are reported in U.S. Pat. Nos. 5,376,383 and 4,976,967.

For subjects requiring neovascularization, in particular, subjects having abnormally elevated apoptotic cell-death of vascular endothelial cells, it may be desirable to administer the mevalonate pathway secondary metabolite agent by a route such as intraarterial administration with clamping, local administration via a balloon catheter, or intraperitoneal injection directly into the affected tissue or wound requiring neovascularization. For example, in the case of intraarterial administration with clamping, the vessel wall in need of such treatment is “isolated” by clamping of the vessel on either side of the “injury” site, resulting in the temporary occlusion of the region to be treated, and allowing local delivery of the mevalonate pathway secondary metabolite agent (e.g., by injection). In the case of intraarterial administration via a balloon catheter, it may be desirable to use a catheter of the “soft-hydrogel surface” type.

Other routes of administration that may be particularly suitable for some subjects (such as, for example, those suffering from a myocardial infarction) include direct intramuscular injection into the myocardium, catheterization of the heart, and intraarterial administration. In some embodiments, intraarterial administration is accompanied with a permeabilizing agent (e.g., nitric oxide), allowing easier access of the mevalonate pathway secondary metabolite agent into the myocardium via the circulation.

In some embodiments, the subject suffers from a wound. In some such embodiments, the mevalonate pathway secondary metabolite agent is applied directly to the wound or to the tissue in the vicinity of the wound.

Pharmaceutical compositions including those already described above may conveniently be presented in unit dosage form and may be prepared by any of a variety of methods known in the art of pharmacy. In general, pharmaceutical compositions are prepared by uniformly and intimately bringing the mevalonate pathway secondary metabolite agent into association with a pharmaceutically acceptable liquid carrier, a pharmaceutically acceptable finely divided solid carrier, or both, and then, optionally, shaping the product.

Delivery systems may optionally include time-release, delayed release or sustained release delivery systems. Such systems can avoid repeated administrations of mevalonate pathway secondary metabolite agent, increasing convenience to the subject and the physician. Many types of release delivery systems are available and known to those of ordinary skill in the art. Examples of delivery systems include, without limitation, polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Non-polymer delivery systems are also suitable for use in accordance with the invention; examples of such systems include, without limitation, lipids including sterols such as cholesterol, cholesterol esters and fatty acids or neutral fats such as mono- di- and tri-glycerides; hydrogel release systems; sylastic systems; peptide based systems; wax coatings; compressed tablets using conventional binders and excipients; partially fused implants; and the like. Specific examples include, but are not limited to, (a) erosional systems in which the 3-hydroxy-3-methylglutaric acid agent is contained in a form within a matrix such as those described in U.S. Pat. Nos. 4,452,775, 4,675,189, and 5,736,152, and (b) diffusional systems in which an active component permeates at a controlled rate from a polymer such as described in U.S. Pat. Nos. 3,854,480, 5,133,974 and 5,407,686. In addition, pump-based hardware delivery systems can be used, some of which are adapted for implantation.

Use of a long-term sustained release implant may be particularly suitable for treatment of chronic conditions. Long-term release, are used herein, means that the implant is constructed and arranged to delivery therapeutic levels of the active ingredient for at least 30 days. In some embodiments, therapeutic levels of active ingredient can be delivered for at least 60 days. Long-term sustained release implants are known to those of ordinary skill in the art and include some of the release systems described above.

Preparations for parenteral administration include sterile aqueous or non-aqueous solutions suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Vehicles for parenteral administration include sodium chloride solution, Ringer's dextrose, dextrose and sodium chloride, lactated Ringer's or fixed oils. Vehicles for intravenous administration include fluid and nutrient replenishers, electrolyte replenishers (such as those based on Ringer's dextrose), and the like. In some embodiments, preservatives and other additives such as, for example, antimicrobials, anti-oxidants, chelating agents, and inert gases and the like, are included.

### F. Combination Therapies

In some embodiments, methods also include administering at least one additional therapy to achieve a desired biological activity. In such combination therapies, two or more different therapeutic agents are administered in overlapping regimens so that the subject is simultaneously exposed to both agents.

Non-limiting examples of suitable agents for use in the one or more additional therapies directed to promoting blood vessel formation and/or vascular function include cytokines, angiogenic growth factors, HMG-CoA reductase inhibitors such as statins described herein, and combinations thereof. Non-limiting examples of angiogenic growth factors include acidic and basic fibroblast growth factor, vascular endothelial growth factor, epidermal growth factor, transforming growth factor, α- and β-platelet derived endothelial cell growth factor, platelet-derived growth factor, tumor necrosis factor α (TNFα), hepatocyte growth factor, insulin-like growth factor, etc.

Non-limiting examples of suitable agents for use in the one or more additional therapies directed to reducing inflammation and/or immunomdulation include anti-inflammatory agents, immunomodulators, immunosuppresive agents, and combinations thereof. Non-limiting examples of anti-inflammatory agents include steroids, non-steroidal anti-inflammatory agents (NSAIDS) (such as, for example, salicylates, fenoprofen, naproxen, piroxicam tolmetin, indomethacin, sulindac, meclofenamate, etc.), and disease modifying anti-rheumatoid drugs (DMARDS) (such as, for example, D-penicillamine, gold salts, hydroxychloroquine, azathioprine, methotrexate, cyclophosphamide, etc.). Non-limiting examples of immunosuppressive agents include cyclosporin A and cyclophosphamide. HMG-CoA reductase inhibitors such as statins mentioned herein may also serve as suitable additional therapies.

### IV. Methods of Diagnosing Using HMG

In certain embodiments, the invention provides methods comprising steps of measuring the amount of mevalonate pathway secondary metabolite agent in a subject; and determining, based on the measurement, that the subject has a disease or condition.

As used herein, the phrase “mevalonate pathway secondary metabolite” refers to a metabolite that is produced secondarily to the inhibition of one or more components in the mevalonate pathway. In some embodiments, the secondary metabolite is produced secondarily to the inhibition of HMG-CoA reductase. For example, the secondary metabolite may be HMG, 2-hydroxyglutarate, 2-oxoglutarate, or a combination thereof. Alternatively or additionally, the secondary metabolite may be one that is identified in screening methods of the invention as discussed above. In some embodiments, amounts of more than one secondary metabolite of HMG-CoA are measured.

A variety of methods are known in the art for measuring amounts of compounds such as secondary metabolites mentioned above. For example, techniques such as gas chromatography-mass spectrometry, high performance liquid chromatography, liquid chromatography-mass spectrometry, gas-liquid chromatography, mass spectrometry, ion chromatography, and enzyme-linked immunosorbent assay can be used alone or in combination to determine relative and/or absolute amounts of compounds. In some embodiments, measurement is conducted in a sample obtained from the subject. For example, biological samples such as biological fluids (such as, for example, blood, lymph, ascites fluid, urine, saliva, synovial fluid, cerebrospinal fluid, vitreous humor, seminal fluid, etc), tissue biopsies, etc. may be used. Tissue and/or cell cultures may also be grown using a biological sample from the subject, and the cultured cells and/or the medium in which such cells grow can be collected for measurements in the practice of the invention. In some embodiments, the amount(s) of secondary metabolites are compared to a control, as described herein.

In general, the disease or condition can be any one in which amounts of mevalonate pathway secondary metabolite is known to be reduced or increased, including diseases and conditions identified in Example 13. Such diseases and conditions may include hypertension, diabetic peripheral vascular disease, gangrene, Buergers syndrome, ischemia, occlusive vascular disease, myocardial infarction, coronary artery disease, visceral artery disease, multiple sclerosis, Alzheimer's disease, atherosclerosis, arthritis, rheumatoid arthritis, sepsis, psoriasis, ischemia, stroke, allograft rejection, type 2 diabetes mellitus, metabolic syndrome, autoimmune disorders, type 1 diabetes mellitus, lupus, peritonitis, reperfusion injury, acquired immunodeficiency syndrome (AIDS), cancer, hyperlipidemia, hypercholesterolemia, and combinations thereof.

In some embodiments, one or more additional diagnostic tools are used together with inventive methods to determine that the subject has a particular condition or disease. Combining inventive diagnostic methods with those known in the art, for example, may enable increased accuracy.

### EXAMPLES

The following examples describe some of the modes of making and practicing the present invention. However, it should be understood that these examples are for illustrative purposes only and are not meant to limit the scope of the invention. Furthermore, unless the description in an Example is presented in the past tense, the text, like the rest of the specification, is not intended to suggest that experiments were actually performed or data were actually obtained.

### Example 1

### Endothelial Cell Proliferation in Response to HMG

It is contemplated that HMG could be a direct mediator of the pleiotropic effects of statins on endothelial cell physiology. The present Example explores this possibility by examining the effect of HMG on endothelial cell proliferation.

Primary endothelial cells (ECs) (such as, for example, human umbilical vein endothelial cells (HUVEC) and human microvascular endothelial cells (HMVEC)) are maintained in culture medium and conditions as specified by the supplier. Endothelial cells are seeded in tissue culture treated 96-well plates at a density of 3×10³ cells/well and incubated in a 37° C. 95/5% air/CO₂ incubator.

To evaluate the effect of HMG on EC proliferation, cells are incubated with HMG in the range from 10 μM to 10 mM for 24 hours. After this 24 hour incubation period, bromodeoxyuridine (BrdU) is added to a final concentration of 10 μM and the cells are allowed to incorporate the BrdU label for 4 hour. BrdU incorporation, an indicator of DNA synthesis and commonly used marker of proliferation, is detected by ELISA according to the manufacturer's instructions (GE Healthcare/Amersham). Vascular endothelial growth factor (VEGF; 100 ng/ml final concentration), pravastatin, and alkaline activated simvastatin are used as positive controls. Organic acids structurally similar to HMG, for example, glutarate and citrate (see FIG. 1), are used to evaluate the specificity of the effects of HMG. In some experiments, the viability of ECs is assessed after exposure to HMG or control agents by trypan blue dye exclusion and/or inner salt (MTS) tetrazolium reduction assay (Promega).

### Example 2

### Effects of HMG on Endothelial Tube Formation

The present Example further explores the effects of HMG on endothelial cell function by evaluating HMG's influence on endothelial tube formation.

Formation of vascular-like structures by endothelial cells (e.g., HUVEC & HMVEC) are performed as described in Kureishi et al. 2000. Chamber well slides are coated with 10 mg/mL Matrigel according to the manufacturer's instructions (BD Biosciences). ECs are seeded on the coated slides at a density of 4×10⁴ cells/well in the media recommended by the supplier and the slides are incubated in a 37° C. 95/5% air/CO₂ incubator. The media is supplemented with HMG, glutarate, citrate (concentration range 10 μM-10 mM), pravastatin, simvastatin, or 100 ng/mL VEGF and the cells incubated for an additional 8-12 h. Tube formation is visualized using an inverted phase contrast microscope with a digital camera. The tube lengths of tubes in random fields are assessed, in a blinded fashion, by image analysis software and data is analyzed by one-way ANOVA with post-hoc testing for statistical significance.

### Example 3

### HMG Effects on Endothelial Cell Migration

The present Example explores another facet of HMG's effects on endothelial cell function by examining HMG's influence on endothelial cell migration.

Endothelial cell migration is evaluated by scratch wound assay (Katsumoto et al. 2005, Urbich et al. 2002, Weis et al. 2001) and by measuring movement of cultured ECs across an extracellular matrix in a modified Boyden chamber assay (Urbich et al. 2002, Auerbach et al. 2003).

### Scratch Wound Assay

ECs (HUVEC & HMVEC) are seeded at 1×10⁵ cell/well in 35 mm×6-well tissue cultured treated plates in the appropriate EC medium. When 70-80% confluency is reached, cells on one-half of the plate—denoted by a line drawn with a marker pen—are scraped away with a sterile cell scraper. Wells are rinsed twice with sterile PBS and then incubated with EC medium supplemented with HMG, glutarate, citrate, VEGF, pravastatin or simvastatin as described in previous Examples. Cell migration is microscopically assessed at 12, 24 and 48 hours and cell numbers in several random fields are determined, in a blinded manner, using image analysis software. Data is analyzed by one-way ANOVA with post-hoc testing to determine statistical significance.

### Modified Boyden Chamber Assay

EC migration response in a Boyden chamber is evaluated in two types of experiments, a pretreatment model and a chemotactic signal model. For the pretreatment approach, ECs are incubated in medium supplemented with HMG glutarate, citrate, VEGF, pravastatin, or simvastatin for 24 hours before being detached and transferred to the upper chamber of a 96-well fibronectin-coated multiwell insert (BD Biosciences) in medium without any test substances. Six or 24 hours after transfer, cells in the lower chamber are stained with Calcein AM according to instructions (BD Bioscience) and fluorescence is measured with a fluorescence microplate reader. Relative fluorescence units are compared across the treatment groups and analyzed by one-way ANOVA with post-hoc testing.

For the chemotactic signal approach, ECs lacking any pretreatment with test substances are transferred to the upper chamber of the BD Biosciences multiwell inserts. The EC medium in the lower chambers will be supplemented with HMG, glutarate, citrate, VEGF, or simvastatin and migration, after 6 or 24 h, will be assessed by Calcein AM staining as described above.

### Example 4

### HMG Production In Vitro Due to Statin Treatment

It is contemplated that HMG mediates some pleiotropic effects of statins, which inhibit HMG-CoA reductase. This idea is consistent with the idea that HMG is a secondary metabolite produced by inhibition of HMG-CoA reductase. The experiments described in this Example and in Examples 5-7 are directed to testing this idea and linking HMG directly to effects of statins.

In the present Example, HMG production in response to statin treatment is tested in endothelial cells. HMG is maintained in the micromolar range in the plasma of healthy adults (Lippe et al. 1987), but the production of HMG in response to statins has never been studied.

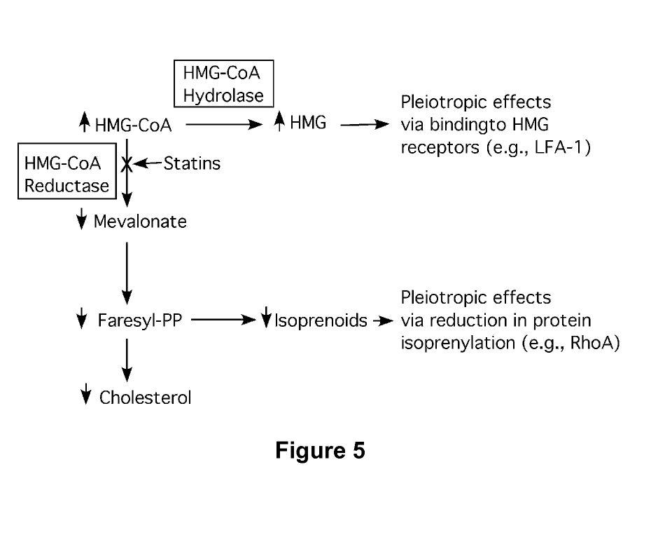

Gas chromatography/mass spectrometry (GC/MS) and high performance liquid chromatography (HPLC) methods (Bjorkman et al. 1976, Chalmers et al. 1989, Lippe et al. 1987) are used to quantify HMG produced secondarily to statin inhibition (see FIG. 5). HMG production by cultured hepatocytes and endothelial cells (ECs) is measured before and after exposure to statins. Pravastatin and simvastatin, as representative hydrophilic and lipophilic type statins respectively, are each clinically important and are employed throughout these studies.

Primary and immortalized (i.e., HepG2) hepatocytes are obtained from Lonza/Clonetics and ATCC, respectively, and maintained in culture according to the supplier's instructions. Primary ECs derived from both large-vessel and microvascular origins (e.g., human umbilical vascular EC (HUVEC) & human microvascular EC (HMVEC; Auerbach et al. 2003), are obtained from Lonza, Cambrex, or a similar vendor and are maintained in culture as specified by the supplier. All EC experiments in this Example are conducted on primary cells of fewer than 10 passages.

HepG2 and endothelial cells are seeded in tissue culture treated 35 mm×6-well plates at a density of 3×10⁶ cells/well and incubated in a 37° C. 95/5% air/CO₂ incubator. Cultured cells are exposed to sodium pravastatin or alkaline activated simvastatin (both obtained from Tocris Bioscience). Tissue culture medium is sampled and cellular extracts prepared as previously described (Bjorkman et al. 1976, Chalmers et al. 1989, Lippe et al. 1987) before and after incubating cells with statins. The concentration of 3-hydroxy-3-methylglutaric acid is measured by both HPLC and GC/MS methods. HMG, succinate, glutaric acid, citrate, and mevalonate are used as HPLC standards Lippe et al. 1987. GC/MS determination of HMG is conducted after samples are derivatized with trimethylsilane as described (Bjorkman et al. 1976, Chalmers et al. 1989). The initial experimental condition exposes cells to pravastatin and simvastatin at a 1 μM concentration for 24 hours. In addition, dose response studies over a statin concentration range of 10 nM to 10 μM and time course studies of a range of 5 min to 48 hours are conducted to fully characterize the secondary metabolic consequences proximal to HMGR. Results are repeated using various statins; doses; time courses; and purification, identification, and quantification techniques for HMG. Key results in HepG2 cells are also repeated using primary human hepatocytes.

### Example 5

### Mevalonate and Isoprenoid Effect on HMG Production

Example 4 tests an alternative hypothesis to the isoprenoid reduction hypothesis of statin pleiotropy. Reversal of pleiotropic effects of statins by mevalonic acid (the product of HMGR) or downstream isoprenoid metabolites has been used as evidence to support the that idea that the mechanism of statin pleiotropy involves a reduction in isoprenoids. Without wishing to be bound by any particular theory, an alternative mechanism for the reversal of statin pleiotropic effects by mevalonic acid or downstream isoprenoid metabolites is proposed. In the proposed alternative mechanism, mevalonic acid or downstream isoprenoid metabolites inhibit the production of HMG by HMG-CoA hydrolase through a feedback regulation pathway.

It is contemplated that HMG promotes angiogenesis and vascular function, which are among the pleiotropic effects of statins. In this Example, mevalonic acid (the product of HMGR) or downstream isoprenoid metabolites are added back and effect on HMG production is measured. Since product feedback inhibition and pathway crosstalk are common features of metabolic pathways, the influence of these compounds on statin-mediated HMG production are examined as well. Although the liver is the primary site of cholesterol production and the primary target organ for statins, direct effects of statins on endothelial cells have been suggested and thus both liver and endothelial cells are examined in this Example.

Mevalonate is maintained in the low nanomolar range in the blood of healthy humans (Parker et al. 1984) but is often added exogenously to reverse the effects of statins at concentrations ranging from 100 μM to 1 mM. To evaluate the potential for mevalonate or mevalonate-derived isoprenoids to influence HMG production, experiments similar to those in Example 4 are conducted in the presence of these compounds. The respective pravastatin and simvastatin concentration and time of exposure yielding optimum HMG production, as identified in Example 4, are used and HepG2 cells. ECs are co-exposed to 0, 10, 100, or 1000 μM mevalonate (after alkaline activation), geranylgeranyl pyrophosphate, or farnesyl pyrophosphate. Culture media and cell extracts are prepared as described herein and HMG production is quantified by HPLC or GC/MS. Key results with HepG2 cells are confirmed with primary human hepatocytes.

Similar feedback experiments can also be performed using exogenously added cholesterol.

### Example 6

### HMG Levels in In Vivo Animal Models

Examples 4-5 test the hypothesis that statin treatment leads to secondary production of HMG in vitro. In this Example, this hypothesis is tested in vivo using animal models. Dose response and time course of HMG production in animals are examined. Furthermore, HMG levels in animal models of disease are examined to determine whether a correlation exists between disease states and HMG levels.

Wild type strains of mice (such as, for example, C57BL/6, BALB/c, 129/SvJ, DBA, etc.), rats, and/or rabbits are treated with statins. HMG content before and after treatment is measured in plasma and urine from treated mice. A variety of doses are employed and HMG content is measured at various times after statin treatment, allowing construction of dose response and time course curves.

HMG levels are also evaluated in animal models of hyperlipidemia (such ApoE knockout mice, LDL receptor null mice, etc.) and diabetes (such as NOD, B6Ins2^{Akita}, B6-db/db, BKS-db/db, etc.). HMG levels are examined in untreated animals to determine whether a correlation exists between disease states and HMG levels. HMG levels are also examined in animals treated with statins to determine whether response to treatment involves HMG production and whether a correlation exists between clinical improvement and HMG production.

### Example 7

### HMG Production Due to Statin Treatment in Humans

HMG levels in plasma and urine samples of human patents (for example, in human volunteers in placebo-controlled clinical trials) can be measured before and after statin treatment to determine if HMG production increases in humans. Statins can also be administered to patients suffering from hyperlipidemia, diabetes, Alzheimer's disease, multiple sclerosis, and other diseases for which statins have unexplained benefits. HMG levels can be measured before and after statin treatment in such patients.

### Example 8

### Effect of HMG on Signaling Through PI3K, Akt, mTOR, and eNOS

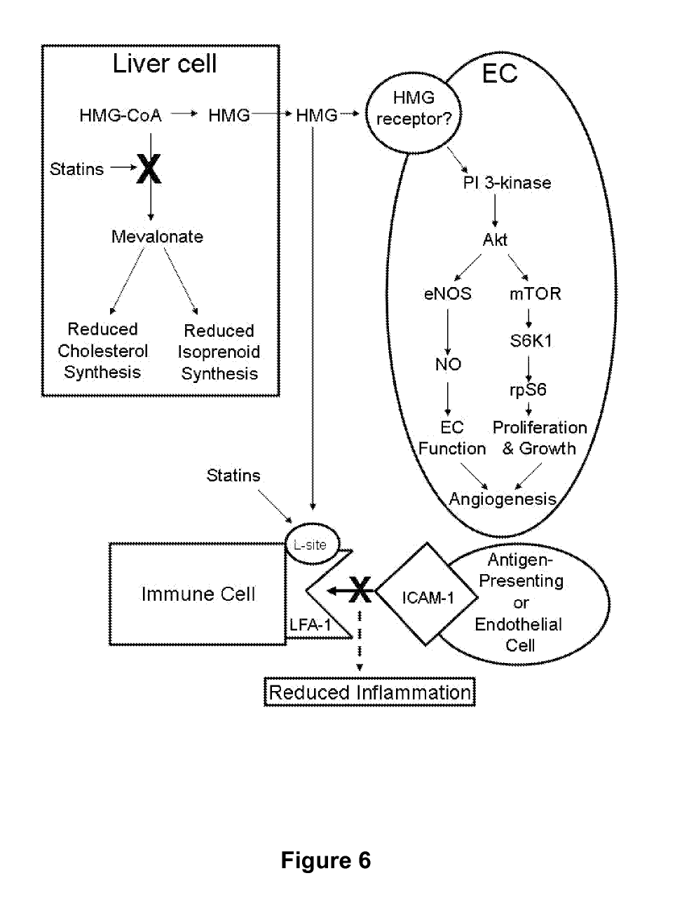

In this Example, we evaluate whether the pro-angiogenic actions of HMG and statins are mediated by similar signal transduction mechanisms. The effect of HMG on known signaling pathways involved in statin pleiotropy is examined. In particular, the effect of HMG on signaling through PI3K, Akt, mTOR and eNOS is evaluated. A variety of investigators have reported rapid effects of statins on endothelial cell signaling involving the growth factor/hormone stimulated kinases phosphoinositide 3-kinase (PI3K), Akt, and mammalian Target of Rapamycin (mTOR), leading to pro-angiogenic behavior and production of nitric oxide (NO) by endothelial nitric oxide synthase (eNOS). In this Example, we examine whether HMG directly activates signaling through these pathways that lead to enhanced EC function (see FIG. 6).

Primary antibodies against total and phospho-Ser 473 Akt, total and phospho-Ser 371 & -Thr389 S6 kinase (S6K1), total and phospho-Ser 240/244 ribosomal protein S6, total and phospho-Ser 1177 & -Ser 615 eNOS, α-tubulin and β-actin are obtained from commercial sources (e.g., Cell Signaling, Santa Cruz, Upstate, etc.). Horseradish peroxidase (HRP)-conjugated secondary antibodies and high sensitivity colorimetric 4CN HRP substrate are purchased from Biorad. LY294002,10-DEBC-HCl, and rapamycin are obtained from commercial sources (e.g., Tocris Bioscience, Biomol, Calbiochem, etc.) and used to inhibit the activity of PI3K, Akt, and mTOR respectively.

For signal transduction pathway studies, ECs (HUVEC & HMVEC) are incubated in basal endothelial cell medium for 4 or 24 hour without serum (Kureishi et al. 2000) prior to stimulation with HMG, VEGF, pravastatin, simvastatin, glutarate, or citrate. In some experiments, ECs are pretreated for 1 hour with LY294002 (10 μM), 10-DEBC-HCl (3 μM), and rapamycin (25 nM) prior to stimulation by HMG, VEGF, or statins. Cell lysates (25 μg total protein) are resolved by 10% SDS-PAGE gels, transferred to PVDF, and immunoblotted for total & phospho forms of Akt, S6K1, S6, and eNOS. Time course studies are conducted by stimulating serum-starved ECs with test agents for 0, 1, 2.5, 5, 10 and 30 minutes. Equivalent protein loading is verified by staining blots for α-tubulin or β-actin. In some experiments, glutarate and citrate (see FIG. 1 for a structure of citrate) are used as structural analogs to evaluate the specificity of HMG's effects.

### Example 9

### Effect of HMG on Nitric Oxide Production by Endothelial Cells

Since eNOS is recognized as a major target through which statins improve EC function and confer vasculoprotective effects, we evaluate the effects of HMG on NO production in this Example.

Time course measurements of EC NO release are conducted by the coupled cGMP method described in Harris et al. 2004. NO-stimulated cGMP production is measured in primary rat aortic smooth muscle cells (RASMCs; passages 3-5; Cell Applications, Inc.). ECs (HUVEC & HMVEC) are incubated in basal endothelial cell medium for 4 or 24 hours without serum (Kureishi et al. 2000) prior to stimulation with HMG, VEGF, pravastatin, simvastatin, glutarate, or citrate. In some experiments, ECs are pretreated for 1 hour with LY294002 (10 μM), 10-DEBC-HCl (3 μM), rapamycin (25 nM), or the eNOS inhibitor L-NIO dihydrochloride (1 μM; Tocris Bioscience) prior to stimulation by HMG, VEGF, or statins. Time course studies are conducted by stimulating serum-starved ECs with test agents for 0, 1, 2.5, 5, 10 and 30 minutes after which time the bathing medium is immediately transferred to 6-well plates confluent with RAMSCs and allowed to react for 3 minutes before being lysed in ice-cold 20 mM sodium acetate, pH 4.0. Samples are stored at −20° C. until cGMP levels are determined using an ELISA kit according to the manufacturer's instructions (Cayman). Glutarate and citrate are used as structural analogs to evaluate the specificity of HMG's effects.

In other experiments, the products of nitric oxide decay (nitrate and nitrite) are measured using the Griess reagent (Molecular Probes) after stimulating ECs 24 hour with HMG, VEGF, statins, or HMG analogs (e.g., glutarate) ±inhibitors.

### Example 10

### Role of PI3K, Akt, mTOR, and eNOS in the Pro-Angiogenic Behavior of Endothelial Cells in Response to HMG

As another means of verifying the involvement of the above signaling elements in the pro-angiogenic responses of ECs, experiments are performed to evaluate the impact of inhibitors of PI3K, Akt, mTOR, and eNOS on EC proliferation (as in Example 1), tube formation (as in Example 2), and migration (as in Example 3). Experiments are conducted as outlined in Examples 2-4 except that ECs are pre-treated for 1 hour with LY294002 (10 μM), 10-DEBC-HCl (3 μM), rapamycin (25 nM), or L-NIO dihydrochloride (1 μM) prior to stimulation by HMG, VEGF, pravastatin, simvastatin, or HMG structural analogs. The inhibitor compounds at the specified concentrations are present during the entire period of stimulation.

### Example 11

### HMG Effects on Inflammation and Immune Function

In addition to promoting angiogenesis and vascular function, statins have other pleiotropic effects including anti-inflammatory effects and immunomodulation. Whether HMG similarly exhibits effects on inflammation and immune function can be examined in vitro. Influence of HMG on Th1/Th2 lymphocyte lineage development; anti-inflammatory properties of HMG related to cytokine production, sepsis, and neuroinflammatory disorders; and effect of HMG on MHC II-mediated antigen presentation can be evaluated. Additionally, direct binding of HMG to lymphocyte function-associated antigen-1 (LFA-1) of immune cells can be evaluated and the effects of such binding on immune cell function and inflammation can be addressed.

### Example 12

### Analysis HMG-CoA Hydrolase and HMG Cell Surface Receptors

To understand further the physiological role of HMG and the link between HMG production and statin therapy, genes for enzymes and cell surface receptors involved in HMG biology can be identified and manipulated.

For example, identification of the gene for HMG-CoA hydrolase will allow further studies that can demonstrate that HMG-CoA hydrolase is the enzyme responsible for producing HMG secondarily to inhibition of HMG-CoA reductase. Based on homology to an enzyme (citrate synthase) whose substrate is similar to HMG, a putative gene for HMG-CoA hydrolase was identified as the one listed under Accession No. XM_{—}293498 in the NCBI database. Nucleic acid sequence information available from the NCBI database will allow direct amplification of the gene by reverse transcription—polymerase chain reaction (RT-PCR) using sequence specific primers and mRNA from HepG2 cells, liver, or other tissues. The amplified gene can be subcloned into bacterial or eukaryotic expression vectors using standard molecular biology techniques and protein expression can be induced. Expressed protein can be assayed for enzymatic activity against HMG-CoA as a substrate. Enzyme assays can use radiolabeled substrates and/or methods such as those described herein to measure HMG.

Once the putative gene is verified to encode an HMG-CoA hydrolase, the gene and/or its product(s) can be thoroughly characterized at the biochemical level (e.g., pH optimum, salt requirement, substrate specificity, substrate kinetics, etc.) and molecular level (e.g., Southern and Northern blotting of various tissues, real time-PCR analysis of tissue expression, etc.) under basal conditions. Transcriptional and translational expression of the gene can also be examined in response to statin treatment in the presence or absence of mevalonate and/or other isoprenoids in the mevalonate pathway.

Genetic manipulation of HMG-CoA hydrolase can facilitate examining the role of HMG in the positive benefits of statins. It is hypothesized, without wishing to be held by theory, that disrupting the function of HMG-CoA hydrolase may eliminate statin pleiotropic effects without affecting cholesterol levels. To demonstrate the role of HMG-CoA hydrolase in mediating some of the pleiotropic effects of statins, gene expression of the gene encoding HMG-CoA hydrolase can be disrupted using techniques such as transgenic approaches, knockout approaches, RNAi (using, for example, small interfering RNAs (siRNA)), etc. For example, the gene encoding HMG-CoA hydrolase can be knocked out in an animal model. Conventional knockouts in which the gene is disrupted throughout the whole body of the animal may be useful, as may conditional knockouts in which gene disruption is restricted to a particular tissue or tissues such as the liver. To verify that activity of HMG-CoA hydrolase is disrupted, knockout cells and/or animals can be assayed for HMG-CoA hydrolase activity.

Once knockout cells and/or animals are verified as null for HMG-CoA hydrolase, they can be used in experiments with statins. For example, levels of cholesterol and triglycerides as well as markers of statin pleiotropic effects (such as, for example, inflammation, T cell activation and differentiation, angiogenesis, etc.) can be monitored in statin-treated HMG-CoA hydrolase-null cells and/or animals can be studied for reduction of Abnormal phenotypes (if any) induced by statin treatment of HMG-CoA hydrolose-null cells and/or animals can be studied. If certain pleiotropic effects are absent or abnormal in null cells and/or animals, HMG can be supplied exogenously to determine if HMG can compensate for (i.e., complement) the loss of HMG-CoA hydrolase activity in the face of statin treatment. Specific signaling pathways (such as, for example, PI-3-kinase/Akt signaling, integrin signaling, and interferon-gamma signaling) can be examined in statin-treated null cells/animals with or without exogenously supplied HMG.

The gene encoding HMG-CoA hydrolase can also or alternatively be overexpressed in cells such HepG2 liver cells. Suitable approaches for overexpression HMG-CoA hydrolase include transfection of cells (using, for example, an adenovirus) and creating of transgenic mice. HMG production by HMG-CoA hydrolase overexpressing cells and/or animals can be measured both in the presence and absence of statins.

To provide further insight into HMG biology, tissue distribution of HMG in animals (whether wild type, null, or overexpressing for HMG CoA-hydrolase) can be studied using radiotracers.

Similar analyses of genes encoding other products involved in HMG biology (such as, for example cell surface receptors for HMG) can also be performed.

### Example 13

### HMG as a Biomarker of Cardiovascular Disease, Inflammation, and Other Disease States

To develop HMG as a biomarker of disease states such as cardiovascular disease and inflammation, HMG levels in longitudinal study designs or in comparisons between healthy and diseased populations can be quantified. Data from this Example will provide critical insight into the physiological function of HMG and may provide improved diagnostic indicators of health, disease, and therapeutic outcome.

### Example 14

### HMG Effects on Inflammatory and Immune Responses

To test whether HMG mediates anti-inflammatory activities and immune system modulation, HMG effects on the synthesis of and signaling by interferon-gamma can be studied. INFγ mediates specific immune and inflammatory responses that could be key factors in the development of atherosclerosis, coronary heart disease, and autoimmunity as well as the inflammatory components of disease such as diabetes, multiple sclerosis, Alzheimer's, and others. Specifically, INFγ mediates the following events that are opposed by statins and, possibly, HMG:

i. increased expression of major histocompatibility complex class II (MHC II) molecules, especially those of professional antigen presenting cells. Increased MHC II expression primes the immune system for a greatly enhanced activation of T cell responses.

ii. increased adhesiveness and infiltration of immune cells by modulating cell surface expression of adhesion molecules such as ICAM-1, LFA-1, CD40, and others.

iii. promotion of the T-helper 2 (Th2) isotype reaction over the T-helper 1 (Th1) isotype reaction.

iv. promotion of nitric oxide (NO) and hydrogen peroxide (H2O2) production.

v. increased activation of the inflammatory transcription factors NF-κB, STAT-1, and IRF-1.

The above IFNγ-mediated events can be studied in HMG-treated animals and/or cells. In vivo and/or in vitro studies can be conducted to examine the influence of HMG on binding interactions between signaling molecules (such as, for example LFA-1/ICAM-1 binding) and subsequent signaling pathways involved in inflammatory and/or immune responses.

### Example 15

### Reduction of Autoimmune and Inflammatory Responses by HMG in an In Vivo Mouse Model

The present Example demonstrates that HMG can be used to ameliorate autoimmune and inflammatory responses in an in vivo mouse model. Experimental autoimmune encephalomyelitis (EAE) was induced in mice. Mice were treated with either HMG, saline, or a positive therapeutic control and evaluated for progression of EAE. Mice treated with HMG exhibited an ameliorated disease phenotype as compared to those injected with saline only, as measured by day of onset of disease and by levels of some cytokines.

### Materials and Methods

### Peptides

PLP₁₃₉₋₁₅₁ is a commercially available encephalitogenic epitope of myelin proteolipid protein (PLP) that has been used to induce EAE in mice.

PLP-BPI (Ac-PLP-BPI-NH₂-2) (Kobayashi et al. 2007), a bifunctional peptide inhibitor, has been shown to exhibit therapeutic effects in a mouse model of EAE. PLP-BPI was synthesized using an automated peptide synthesis system for use as a positive control. After cleavage from the resin, PLP-BPI peptide was purified by reversed-phase HPLC using a C₁₈ column. The purity of the peptide was analyzed using an analytical C₁₈ column. The identity of the synthesized peptide was confirmed by matrix-assisted laser desorption/ionization-time of flight mass spectrometry as well as LC/MS-MS electrospray ionization with a positive ion mode.

### Induction of EAE

To induce EAE, five-to-seven week old SJL/J (H-2S) female mice (Charles River; Wilmington, Mass.) were immunized subcutaneously with 200 μg PLP₁₃₉₋₁₅₁ in a 0.2 mL emulsion comprised of equal volumes of phosphate-buffered saline (PBS) and complete Freund's adjuvant (CFA) containing killed Mycobacterium Tuberculosis strain H37RA (at a final concentration of 4 mg/ml, Difco, Detroit, Mich.). PLP₁₃₉₋₁₅₁/CFA was administered to regions above the shoulder and the flanks (4 sites total; 50 μL at each injection site). In addition, 200 ng of pertussis toxin (List Biological Laboratories, Campbell, Calif.) was injected intraperitoneally on the day of immunization and 2 days post immunization.

### Treatment and Clinical Assessment

Mice received intravenous injections of vehicle (PBS), HMG (25 μmol/mouse), or PLP-BPI (100 nmol/mouse) on days 4, 7, and 10 (n=6 per treatment group). Disease progression was evaluated blindly using a clinical scoring as follows: 0—no clinical symptoms, 1—tail weakness or limp tail, 2—paraparesis (weakness or incomplete paralysis of one or two hind limbs, 3—paraplegia (complete paralysis of two hind limbs), 4—paraplegia with forelimb weakness or paralysis, and 5—moribund (mice were euthanized once they were found to be moribund). Body weight was also measured daily.

### Cytokine Measurements

Blood samples were collected from the facial vein in PBS-, HMG-, and PLP-BPI-treated mice on day 14 and day 28 (n=3 per group). Blood samples were allowed to clot overnight at 2-8° C. before centrifuging for 10 min at 2000×g. Serum was removed and stored at −80° C. until measurement of cytokines and chemokines was performed by a commercial analytical services laboratory.

The levels of key cytokines and chemokines were measured by multiplex ELISA array by a commercial analytical laboratory.

### Results and Discussion

HMG-treated mice exhibited an ameliorated disease phenotype as compared to those treated with vehicle only. The average day of onset of EAE in HMG-treated mice was delayed by about 2 days as compared to PBS treated mice. Day of onset is considered to be the day when the EAE clinical score is one or more, which occurred on days 11, 13, and 18 for PBS-, HMG-, and PLP-BPI-treated SJL/J mice respectively.

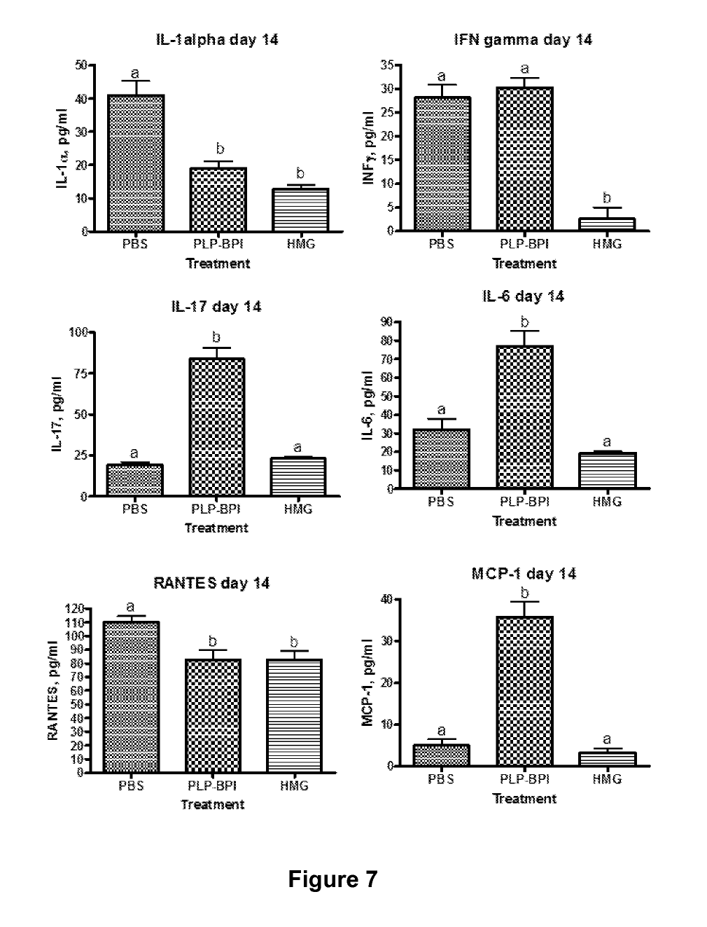

HMG-treated mice also showed reduced inflammation as measured by levels of some cytokines. As shown in FIG. 7, animals treated with PBS only had statistically significant elevations in interleukin 1-alpha (IL-1 alpha), interferon-gamma (IFN gamma), and RANTES compared to animals treated with HMG.

Interleukin-1 is a key regulatory mediator of inflammation in neurodegenerative disease and may act by directly causing local inflammation that damages neurons and by helping to increase the permeability of the blood-brain barrier to allow invasion of inflammatory cells of the innate and adaptive immune systems (John et al., 2005; Simi et al., 2007; Patel et al., 2003). IL-1 alpha levels were also significantly decreased in animals treated with the experimental therapeutic molecule PLP-BPI, which has been shown to effectively suppress EAE disease responses (Kobayashi et al., 2007).

Central nervous system inflammation and damage in EAE occur when primed T cells cross the blood-brain barrier and are reactivated by MHC class II antigen presenting cells (Goverman, 2009, Mills, 2008). CD4+ T cells of the TH1 and TH17 subtypes, which secrete IFN gamma and IL-17 respectively, are primary effector cells involved in the pathology of EAE (Goverman, 2009). HMG-treated animals had much lower serum concentrations of IFN gamma than either PBS- or PLP-BPI-treated mice. In addition, HMG-treated animals had significantly lower levels of IL-17 compared to PLP-BPI-treated animals. IL-6, a pro-inflammatory mediator that TH17 cells both directly synthesize and induce in other cell types, was significantly increased in PLP-BPI-treated mice compared to PBS- and HMG-treated groups.

RANTES, also known as CCL5, is a chemokine produced by circulating T cells and which is induced by IL-1 alpha. RANTES is chemotactic for T-cells, human eosinophils and basophils and plays an active role in recruiting leukocytes into inflammatory sites (http://www.copewithcytokines.de/cope.cgi?key=RANTES). Both HMG- and PLP-BPI-treated mice showed statistically significant reductions in IL-1 alpha and RANTES compared to the PBS treatment group.

MCP-1, also called CCL2, is a chemokine that recruits monocytes, memory T cells, and dendritic cells to sites of tissue injury and infection. Compared to the PBS- and HMG-treated groups, mice treated with the PLP-BPI peptide showed significant elevations of MCP-1.

### Example 16

### Effects of HMG on Angiogenesis and Vascular Function

The present Example describes experiments in a mouse hindlimb ischemia model that can be used to examined the effects of HMG on angiogenesis and vascular function.

Previous studies on statins and vascular function have focused on the effects of statins in an acute ischemia-reperfusion setting. Only a few studies have used a model of chronic ischemia such as hindlimb ischemia. Such studies of statins in hindlimb injury have shown that statins promote recovery of blood flow and neovascularization (Kureishi et al. 2000, Sata et al. 2004, and Shimada et al. 2004).

We hypothesize that HMG is a secondary metabolite produced secondarily to the inhibition of HMG-CoA reductase by statins. To determine whether HMG can also promote revascularization and vascular function, HMG is tested in a hindlimb ischemia model.

Hindlimb ischemia is induced as follows: C57BL/6 mice are anesthetized, placed on a thermal pad, and prepared for surgery. An incision is made along the right hind leg. The four branches of the femoral artery are ligated with sterile suture and the vasculature is excised. The incision is closed with veterinary glue.

Either saline or HMG (20 mg/kg body weight per day) is injected intraperitoneally for five consecutive days starting on the day of the surgery. Five mice are injected with saline, and six mice are injected with HMG.

To assess re-vascularization and vascular function, laser Doppler perfusion imaging is performed on days 0, 4, 7, and 15 after surgery. The ratio of blood flow in the right hind leg (ischemic) to that of the left hind leg (non-ischemic) is calculated for each time point analyzed. Data is analyzed by two-way analysis of variance to determine statistical significance.

### REFERENCES

Unless otherwise stated, the contents of all references (including patents, patent publications, and non-patent literature) mentioned herein, including the references listed below, are incorporated by reference herein in their entirety.
Auerbach R, Lewis R, Shinners B, Kubai L, Akhtar N. Angiogenesis assays: a critical overview. Clin Chem 2003; 49: 32-40.
Baran et al. 1985. “3-Alkyl-3-hydroxyglutaric Acids: A New Class of Hypocholesterolemic HMG CoA Reductase Inhibitors.” J. Med. Chem. 28:597-601
Bjorkman L, McLean C, Steen G. Organic acids in urine from human newborns. Clin Chem 1976; 22: 49-52.
Capoccia B J, Shepherd R M, Link D C. G-CSF and AMD3100 mobilize monocytes into the blood that stimulate angiogenesis in vivo through a paracrine mechanism. Blood. 2006; 108: 2438-45.
Chalmers R A, Tracey B M, Mistry J, Stacey T E, McFadyen I R. Prenatal diagnosis of 3-hydroxy-3-methylglutaric aciduria by GC-MS and enzymology on cultured amniocytes and chorionic villi. J Inher Metab Dis 1989; 12: 286-292.
Goverman, J. 2009. Autoimmune T cell responses in the central nervous system. Nature Rev. Immunol. 9:393-407.
Harris M B, Blackstone M A, Sood S G, Li C, Goolsby J M, Venema V J, Kemp B E, Venema R C. Acute activation and phosphorylation of endothelial nitric oxide synthase by HMG-CoA reductase inhibitors. Am J Physiol 2004; 287: H560-H566.
Houten et al., Regulation of isoprenoid/cholesterol biosynthesis in cells from mevalonate kinase-deficient patients. J. Biol. Chem. 2003; 278 (8): 5736-5743
John G R, Lee S C, Song X, Rivieccio M, Brosnan C F. 2005. IL-1-regulated responses in astrocytes: relevance to injury and recovery. Glia 49:161-76.
Jones M G and Chalmers R A. Artefacts in organic acid analysis: occurrence and origin of partially trimethylsilylated 3-hydroxy-3-methyl carboxylic acids. Clinica Chimica Acta. 2000; 300:203-212
Katsumoto M, Shingu T, Kuwashima, R, Nakata A, Nomura S, Chayama K. Biphasic effect of HMG-CoA reductase inhibitor, pitavastatin, on vascular endothelial cells and angiogenesis. Circulation J 2005; 69: 1547-55.
Kobayashi N, Kobayashi H, Gu L, Malefyt T, Siahaan T J. 2007. Antigen-specific suppression of experimental autoimmune encephalomyelitis by a novel bifunctional peptide inhibitor. J. Pharmacol. Exper. Therap. 322:879-86.
Kureishi Y, Luo Z, Shiokima I, Bialik A, Fulton D, Lefer D J, Sessa W C, Walsh K. The MG-CoA reductase inhibitor simvastatin activates the protein kinase Akt and promotes angiogenesis in normocholesterolemic animals. Nature Med 2000; 6: 1004-10.
Lippe G, Trevisan R, Nosadini R, Fabris R, Deana R. 3-hydroxy-3-methylglutaric acid, adipic, and 2-oxoglutaric acids measured by HPLC in the plasma from diabetic patients. Clin Biochem 1987; 20: 275-9.
Mills, K H G. 2008. Induction, function and regulation of IL-17-producing T cells. Eur. J. Immunol. 38:2636-49.
Parker T S, McNamara D J, Brown C D, Kolb R, Ahrens E H Jr, Alberts A W, Tobert J, Chen J, De Schepper P J. Plasma mevalonate as a measure of cholesterol synthesis in man. J Clin Invest 1984; 74: 795-804.
Patel H C, Boutin H, Allan S M. 2003. Interleukin-1 in the brain: mechanisms of action in acute neurodegeneration. Ann. N.Y. Acad. Sci. 992:39-47.
Prietsch et al., Mevalonate kinase deficiency: enlarging the clinical and biochemical spectrum. Pediatrics; 2003; 111 (2):258-261
Sata M, Nishimatsu H, Osuga J-I, Tanaka K, Ishizaka N, Ishibashi S, Hirata Y, Nagai R. Statins augment collateral growth in response to ischemia but they do not promote cancer and atherosclerosis. Hypertension. 2004; 43: 1214-1220.
Simi A, Tsakiri N, Wang P, Rothwell N J. Interleukin-1 and inflammatory neurodegeneration. Biochem. Soc. Trans. 2007; 35:1122-26.

### Other Embodiments

Other embodiments of the invention will be apparent to those skilled in the art from a consideration of the specification or practice of the invention disclosed herein. It is intended that the specification and examples be considered as exemplary only, with the true scope of the invention being indicated by the following claims.

What is claimed is:

1. A method comprising steps of:
(a) applying at least one inhibitor of at least one component in the mevalonate pathway to a system comprising cells; and
(b) identifying compounds whose amounts are higher in the absence of the inhibitor as compared to in its presence.

2. The method of claim 1, further comprising a step of testing compounds identified in step (b) for a biological activity.

3. The method of claim 2, wherein the biological activity is a pleiotropic effect of statins.

4. The method of claim 1, wherein the component is HMG-CoA reductase.

5. The method of claim 4, wherein the inhibitor is a statin molecule.

6. The method of claim 5, wherein the statin molecule is selected from the group consisting of lovastatin, pravastatin, simvastatin, fluvastatin, atorvastin, rosuvastatin, cerivastatin, compactin, NK-104, and combinations thereof.

7. The method of claim 1, wherein the cells comprise hepatocytes.

8. The method of claim 1, wherein the step of identifying comprises performing a technique selected from the group consisting of gas chromatography-mass spectrometry, high performance liquid chromatography, liquid chromatography-mass spectrometry, gas-liquid chromatography, mass spectrometry, and ion chromatography.

9. A method comprising steps of:
applying at least one HMG-CoA reductase inhibitor to a system comprising cells; and
obtaining 3-hydroxy-3-methylglutaric acid (HMG) from the system.

10. The method of claim 9, wherein the at least one HMG-CoA reductase inhibitor is a statin molecule.

11. The method of claim 10, wherein the statin molecule is selected from the group consisting of lovastatin, pravastatin, simvastatin, fluvastatin, atorvastin, rosuvastatin, cerivastatin, compactin, NK-104, and combinations thereof.

12. The method of claim 9, wherein the cells comprise hepatocytes.

13. A method comprising a step of:
administering a mevalonate pathway secondary metabolite agent to a subject in an amount effective such that extent of blood vessel formation or vascular function is increased as compared with that observed or expected for a control subject to whom mevalonate pathway secondary metabolite agent is not administered.

14. The method of claim 13, wherein the mevalonate pathway secondary metabolite agent is a 3-hydroxy-3-methylglutaric acid agent.

15. The method of claim 14, wherein the 3-hydroxy-3-methylglutaric acid agent is 3-hydroxy-3-methylglutaric acid (HMG).

16. The method of claim 13, wherein angiogenesis, vasculogenesis, or both are increased.

17. The method of claim 13, wherein vascular function is increased.

18. The method of claim 17, wherein the vascular function is selected from the group consisting of blood flow, vasodilation, nitric oxide production, and combinations thereof.

19. The method of claim 13, wherein the subject is nonhyperlipidemic.

20. The method of claim 13, wherein the subject is nonhypercholesterolemic.

21. The method of claim 13, wherein the subject is suffering from a condition selected from the group consisting of hypertension, diabetic peripheral vascular disease, gangrene, Buerger's syndrome, ischemia, occlusive vascular disease, obstructive vascular disease, peripheral vascular disease, myocardial infarction, coronary artery disease, visceral vascular disease, and combinations thereof.

22. The method of claim 21, wherein the subject is suffering from an ischemia selected from the group consisting of ischemia of the muscle, ischemia of the brain, ischemia of the kidney, ischemia of the lung, ischemia of the heart (myocardial ischemia), ischemia of the limb, ischemia islets of Langerhans cells, and combinations thereof.

23. The method of claim 13, wherein the subject has a wound.

24. The method of claim 13, wherein the mevalonate pathway secondary metabolite agent is administered by a route selected from the group consisting of topical, enteral, parenteral, epidural, and intravitreal.

25. The method of claim 24, wherein the mevalonate pathway secondary metabolite agent is administered orally.

26. The method of claim 24, wherein the mevalonate pathway secondary metabolite agent is administered by a parenteral route selected from the group consisting of intravenous, intraarterial, intramuscular, intracardiac, subcutaneous, intraosseous, intradermal, intrathecal, intraperitoneal, transdermal, transmucosal, inhalational, and infusion.

27. The method of claim 13, wherein the mevalonate pathway secondary metabolite agent is administered locally to a tissue for which angiogenesis is desired.

28. The method of claim 27, wherein the step of administering comprises inserting a stent comprising mevalonate pathway secondary metabolite agent into the tissue.

29. The method of claim 13, wherein the step of administering comprises administering a pharmaceutical composition comprising mevalonate pathway secondary metabolite agent and a pharmaceutically acceptable carrier.

30. The method of claim 29, wherein the pharmaceutical composition is suitable for topical application.

31. The method of claim 30, wherein the pharmaceutical composition comprises a salve, a gel, a film, a patch, or a combination thereof.

32. A method comprising a step of
administering to a subject suffering from or susceptible to a disease or condition selected from the group consisting of an inflammatory disease or condition, a condition for which immunomodulation is desired, or both an amount of mevalonate pathway secondary metabolite agent effective to ameliorate the disease or condition and/or modulate the immune system.

33. The method of claim 32, wherein the subject is suffering from a disease or condition selected from the group consisting of acquired immunodeficiency syndrome (AIDS), allograft rejection, Alzheimer's disease, arthritis, atherosclerosis, an autoimmune disorder, cancer, ischemia, metabolic syndrome, multiple sclerosis, peritonitis, sepsis, stroke, type 2 diabetes mellitus, reperfusion injury, and combinations thereof.

34. The method of claim 33, wherein the disease or condition is an autoimmune disorder selected from the group consisting of Addison's disease, autoimmune hepatitis Celiac disease, Crohn's Disease, giant cell arteritis, Goodpasture's syndrome, Grave's disease, Guillain-Barre syndrome, Hashimoto's thyroiditis, juvenile rheumatoid arthritis, lupus, polymyalgia rheumatica, psoriasis, primary biliary cirrhosis, rheumatoid arthritis, scleroderma, sclerosing cholangitis, Sjogren's syndrome, temporal arteritis, type 1 diabetes mellitus, ulcerative colitis, Wegener's granulomatosis, and combinations thereof.

35. A method comprising a step of:
administering to cells a mevalonate pathway secondary metabolite agent and
administering the cells to a subject in need of cell therapy.

36. The method of claim 35, wherein the cells comprise stem cells or progenitor cells.

37. The method of claim 35, wherein the cells comprise islet of Langerhans cells.

38. The method of claim 35, wherein the mevalonate pathway secondary metabolite agent enhances mobilization, migration, or homing of the cells.

39. The method of claim 35, wherein the mevalonate pathway secondary metabolite agent enhances proliferation or differentiation the cells.

40. The method of claim 35, wherein the mevalonate pathway secondary metabolite agent enhances engraftment or revascularization of the cells.

41. The method of claim 35, wherein the mevalonate pathway secondary metabolite agent prevents or reduces host immune-inflammatory responses to the cells.

42. A method comprising steps of:
measuring the amount of a mevalonate pathway secondary metabolite in a subject; and
determining, based on the measurement, that the subject has a disease or condition.

43. The method of claim 42, wherein the secondary metabolite is selected from the group consisting of HMG, 2-hydroxyglutarate, and 2-oxoglutarate, and combinations thereof.

44. The method of claim 42, wherein the secondary metabolite is selected from the group consisting of HMG, 2-hydroxyglutarate, and 2-oxoglutarate, and combinations thereof.

45. The method of claim 42, wherein the secondary metabolite is HMG.

46. The method of claim 42, wherein the disease or condition is selected from the group consisting of hypertension, diabetic peripheral vascular disease, gangrene, Buergers syndrome, ischemia, occlusive vascular disease, myocardial infarction, coronary artery disease, visceral artery disease, multiple sclerosis, Alzheimer's disease, atherosclerosis, arthritis, rheumatoid arthritis, sepsis, psoriasis, ischemia, stroke, allograft rejection, type 2 diabetes mellitus, metabolic syndrome, autoimmune disorders, type 1 diabetes mellitus, lupus, peritonitis, reperfusion injury, acquired immunodeficiency syndrome (AIDS), cancer, and combinations thereof.

47. The method of claim 42, wherein the step of measuring comprises performing a technique selected from the group consisting of gas chromatography-mass spectrometry, high performance liquid chromatography, liquid chromatography-mass spectrometry, gas-liquid chromatography, mass spectrometry, and ion chromatography.

48. The method of claim 42, where in the step of determining comprises determining that the amount of the secondary metabolite is increased as compared to that expected or observed of a control subject.
